(19) United States
(12) Patent Application Publication
(10) Pub. No.: US 2024/0016933 A1
(43) Pub. Date: Jan. 18, 2024

(54) Methods of Anchoring or Reconstituting Active Molecules on Metabolically Labeled Cells

(71) Applicant: The General Hospital Corporation, Boston, MA (US)

(72) Inventors: James T. Kurnick, Quincy, MA (US); Ian Seymour Dunn, Beverly, MA (US); Matthew M. Lawler, Madison, WI (US)

(21) Appl. No.: 18/036,047

(22) PCT Filed: Nov. 5, 2021
(86) PCT No.: PCT/US2021/058295

§ 371 Date: May 9, 2023

Related U.S. Application Data
(60) Provisional application No. 63/111,188, filed on Nov. 9, 2020.

(51) Int. Cl. A61K 39/00; C07K 16/10; C12N 5/0783; C07H 15/12; C07K 16/28; C07K 16/30; A61K 35/17

(52) U.S. Cl. CPC A61K 39/4631; C07K 16/1018; C12N 5/0636; C07H 15/12; C07K 16/2803; C07K 16/3015; C07K 16/1027; A61K 35/17; C07K 16/2863; A61K 2239/13

(57) ABSTRACT

The present disclosure provides methods of anchoring active molecules on the surface of a cell, methods of anchoring at least two active molecules on the surface of a cell, and methods of enhancing an immune response to a target cell in a human.

### CROSS-REFERENCE TO RELATED APPLICATIONS

This application claims priority to U.S. Provisional Patent Application No. 63/111,188, filed Nov. 9, 2020. The contents of this application are incorporated herein by reference in its entirety.

### FIELD OF THE INVENTION

The present disclosure is directed, in part, to methods of anchoring active molecules on the surface of a cell, methods of anchoring at least two active molecules on the surface of a cell, and methods of enhancing an immune response to a target cell in a human.

### BACKGROUND

Numerous therapeutic antibodies, or antigen-binding fragments thereof, are currently being used in the treatment of various diseases and conditions. Additional treatment regimens for using these same therapeutic antibodies, or antigen-binding fragments thereof, to treat different diseases and conditions is needed. In addition, numerous chimeric antigen receptor (CAR) T (CAR-T) cells are currently being used or studied in the treatment of various diseases and conditions. Additional treatment regimens for using these same CAR-T cells to treat different diseases and conditions is needed.

### SUMMARY

The present disclosure provides methods of anchoring an active molecule on the surface of a cell, the method comprising the steps: a) contacting the cell with an azide-modified sugar; and b) contacting the cell with an active molecule conjugated to an azide reactive molecule, wherein the azide reactive molecule is chemically reactable with the azide of the azide-modified sugar.

The present disclosure also provides methods of anchoring at least two active molecules on the surface of a cell, the method comprising the steps: a) contacting the cell with an azide-modified sugar; b) contacting the cell with a first active molecule conjugated to a first azide reactive molecule, wherein the first azide reactive molecule is chemically reactable with the azide of the azide-modified sugar; and c) contacting the cell with a second active molecule conjugated to a second azide reactive molecule, wherein the second azide reactive molecule is chemically reactable with the azide of the azide-modified sugar.

The present disclosure also provides methods of anchoring an active molecule on the surface of a cell, the method comprising the steps: a) contacting the cell with an azide-modified sugar; b) contacting the cell with a first small interactive peptide conjugated to an azide reactive molecule, wherein the azide reactive molecule is chemically reactable with the azide of the azide-modified sugar; and c) contacting the cell with a second small interactive peptide conjugated to an active molecule, wherein the first small interactive peptide interacts with the second small interactive peptide.

The present disclosure also provides methods of enhancing an immune response to a target cell in a human, the method comprising the steps: a) contacting the target cell in the human with an azide-modified sugar; b) introducing into the human a CAR-T cell, wherein the CAR-T cell comprises an extracellular FK506-binding protein (FKBP) domain or FRB domain functionally linked to a cytoplasmic signaling domain of the CART-T cell; and c) contacting the target cell in the human with a bifunctional FKBP or FRB domain binding compound, wherein a first portion of the bifunctional FKBP domain binding compound interacts with the FKBP domain on the CAR-T cell, and a second portion of the bifunctional FKBP or FRB domain binding compound interacts with the azide of the azide-modified sugar on the target cell.

### BRIEF DESCRIPTION OF THE DRAWINGS

FIG. 1 shows a representation of the placement of single (upper panel) or dual epitopes (lower panel) on metabolically-labeled cells.

FIG. 2 shows a representation of a flow cytometry analysis of the placement of a mimotope for Trastuzumab on a cell surface by azide metabolic labeling and treatment with DBCO-mimotope, followed by recognition via the cognate antibody, and a secondary anti-human kappa antibody.

FIG. 3 shows a representation of a CAR-T system comprising an extracellular FKBP domain functionally linked to a cytoplasmic signaling and activation domain within the CART-T cell.

FIG. 4 shows a representation of a CAR-T system for targeting surface azide-modified cells.

### DETAILED DESCRIPTION

The terminology used herein is for the purpose of describing particular embodiments only and is not intended to be limiting. The terms used in this disclosure adhere to standard definitions generally accepted by those having ordinary skill in the art. In case any further explanation might be needed, some terms have been further elucidated below.

As used herein, the singular forms “a,” “an,” and “the” include plural references unless the content clearly dictates otherwise.

As used herein, the term “alkyl” means a saturated hydrocarbon group which is straight-chained or branched. In some embodiments, the alkyl group has from 1 to 20 carbon atoms, from 2 to 20 carbon atoms, from 2 to 16 carbon atoms, from 4 to 12 carbon atoms, from 4 to 16 carbon atoms, from 4 to 10 carbon atoms, from 1 to 10 carbon atoms, from 2 to carbon atoms, from 1 to 8 carbon atoms, from 2 to 8 carbon atoms, from 1 to 6 carbon atoms, from 2 to 6 carbon atoms, from 1 to 4 carbon atoms, from 2 to 4 carbon atoms, from 1 to 3 carbon atoms, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, octyl, nonyl, 4,4 dimethylpentyl, decyl, undecyl, dodecyl, 2,2,4-trimethylpentyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the terms “bind,” “binds,” “binding,” and “bound” refer to a stable interaction between two molecules that are close to one another. The terms include physical interactions, such as chemical bonds (either directly linked or through intermediate structures), as well as non-physical interactions and attractive forces, such as electrostatic attraction, hydrogen bonding, and van der Waals/dispersion forces.

As used herein, the terms “comprising” (and any form of comprising, such as “comprise”, “comprises”, and “comprised”) and “having” (and any form of having, such as “have” and “has”) are inclusive and open-ended and include the options following the terms, and do not exclude additional, unrecited elements, or method steps.

As used herein, the term “contacting” means bringing together a compound and a cell, or a compound with another compound in an in vitro system or an in vivo system.

As used herein, the phrase “ethylene glycol unit” means a polymer of —(O—CH₂—CH₂)ₙ—O—, wherein n is from 1 to about 20. A polyethylene glycol (PEG) having 4 ethylene glycol units (i.e., —(O—CH₂—CH₂)₄—O—) is referred to herein as PEG4.

At various places herein, substituents of compounds may be disclosed in groups or in ranges. Designation of a range of values includes all integers within or defining the range (including the two endpoint values), and all subranges defined by integers within the range. It is specifically intended that the disclosure include each and every individual subcombination of the members of such groups and ranges. For example, the term “C₁₋₆alkyl” is specifically intended to individually disclose methyl, ethyl, propyl, C₄alkyl, C₅alkyl, and C₆alkyl.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

The structures depicted herein may omit necessary hydrogen atoms to complete the appropriate valency. Thus, in some instances a carbon atom or nitrogen atom may appear to have an open valency (i.e., a carbon atom with only two bonds showing would implicitly also be bonded to two hydrogen atoms; in addition, a nitrogen atom with a single bond depicted would implicitly also be bonded to two hydrogen atoms). For example, “—N” would be considered by one skilled in the art to be “—NH₂.” Thus, in any structure depicted herein wherein a valency is open, one or more hydrogen atoms, as appropriate, is implicit, and is only omitted for brevity.

The compounds described herein can also include all isotopes of atoms occurring in the intermediates or final compounds. Isotopes include those atoms having the same atomic number but different mass numbers. For example, isotopes of hydrogen include tritium and deuterium. Carbon (¹²C) can be replaced at any position with ¹³C or ¹⁴C. Nitrogen (¹⁴N) can be replaced with ¹⁵N. Oxygen (¹⁶O) can be replaced at any position with 170 or 180. Sulfur (³²S) can be replaced with ³³S, ³⁴S or ³⁶S. Chlorine (³⁵Cl) can be replaced with ³⁷Cl. Bromine (⁷⁹Br) can be replaced with ⁸¹Br.

In some embodiments, the compounds, or salts thereof, are substantially isolated. Partial separation can include, for example, a composition enriched in any one or more of the compounds described herein. Substantial separation can include compositions containing at least about 50%, at least about 60%, at least about 70%, at least about 80%, at least about 90%, at least about 95%, at least about 97%, or at least about 99% by weight of any one or more of the compounds described herein, or salt thereof. Methods for isolating compounds and their salts are routine in the art.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Contacting Cells with Azide-Modified Sugars

The present disclosure provides methods whereby cells are contacted with an azide-modified sugar. In any of the embodiments described herein, the cell can be any desired target cell. In some embodiments, the cell is a virus infected cell, a tumor cell, a cell infected with a microbe, or a cell that produces a molecule that leads to a disease, such as a cell that produces an antibody that induces allergy, anaphylaxis, or autoimmune disease, or a cytokine that mediates a disease. The cells can be cells of the immune system that are contributing to autoimmunity such as cells of the adaptive or innate immune systems, transplant rejection, or an allergic response. The cells described herein can be contacted with any of the azide-modified sugars described herein either in vitro or in vivo.

In some embodiments, the cell is a tumor cell or cancer cell. Representative tumor or cancer cells include, but are not limited to: acute lymphoblastic leukemia (ALL), acute myeloid leukemia (AML), adrenocortical carcinoma, Kaposi sarcoma, lymphoma, anal cancer, astrocytoma, atypical teratoid/rhabdoid tumor, basal cell carcinoma, bile duct cancer, bladder cancer, bone cancer (Ewing sarcoma, osteosarcoma, and malignant fibrous histiocytoma), brain tumor, breast cancer, bronchial tumor, Burkitt lymphoma, non-Hodgkin lymphoma, carcinoid tumor, cardiac tumor, embryonal tumor, germ cell tumor, cervical cancer, cholangiocarcinoma, chordoma, chronic lymphocytic leukemia (CLL), chronic myelogenous leukemia (CML), chronic myeloproliferative neoplasm, colorectal cancer, craniopharyngioma, cutaneous T-cell lymphoma (mycosis fungoides and Sézary syndrome), ductal carcinoma in situ (DCIS), embryonal tumor, endometrial cancer, uterine cancer, ependymoma, esophageal cancer, esthesioneuroblastoma, extracranial germ cell tumor, extragonadal germ cell tumor, eye cancer, childhood intraocular melanoma, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumor (GIST), testicular cancer, gestational trophoblastic disease, hairy cell leukemia, head and neck cancer, hepatocellular cancer, histiocytosis, Hodgkin lymphoma, hypopharyngeal cancer, islet cell tumors, pancreatic neuroendocrine tumor, kidney (renal cell) cancer, laryngeal cancer, papillomatosis, lip and oral cavity cancer, liver cancer, lung cancer (non-small cell and small cell), male breast cancer, Merkel cell carcinoma, mesothelioma, malignant childhood mesothelioma, metastatic cancer, metastatic squamous neck cancer, midline tract carcinoma, mouth cancer, multiple endocrine neoplasia syndrome, multiple myeloma/plasma cell neoplasm, myelodysplastic syndrome, myelodysplastic/myeloproliferative neoplasm, nasal cavity and paranasal sinus cancer, nasopharyngeal cancer, neuroblastoma, oral cancer, ovarian cancer, pancreatic cancer, paraganglioma, parathyroid cancer, penile cancer, pharyngeal cancer, pheochromocytoma, pituitary tumor, plasma cell neoplasm, pleuropulmonary blastoma, primary peritoneal cancer, prostate cancer, rectal cancer, rhabdomyosarcoma, salivary gland cancer, vascular tumor, uterine sarcoma, small intestine cancer, soft tissue sarcoma, squamous cell carcinoma, stomach cancer, T-cell lymphoma, testicular cancer, throat cancer, thymoma, thymic carcinoma, thyroid cancer, transitional cell cancer of the renal pelvis and ureter, urethral cancer, vaginal cancer, and Wilms tumor. Each of these types of cancer cells can be labeled with any of the azide-modified sugars described herein, to generate an epitope for a therapeutic molecule, such as an antibody, or antigen-binding fragment thereof.

In any of the embodiments described herein, the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA). In some embodiments, the azide-modified sugar is AzNAM. In some embodiments, the azide-modified sugar is AzGlcNAc. In some embodiments, the azide-modified sugar AGalNAc. In some embodiments, the azide-modified sugar is AzNANA. In any of the embodiments described herein, the azide-modified sugar is acetylated at 1, 2, 3, or 4 positions. In some embodiments, the azide-modified sugar is acetylated at 1 position. In some embodiments, the azide-modified sugar is acetylated at 2 positions. In some embodiments, the azide-modified sugar is acetylated at 3 positions. In some embodiments, the azide-modified sugar is acetylated at 4 positions.

### Methods of Anchoring Active Molecules

The present disclosure provides methods of anchoring an active molecule on the surface of a cell. These methods comprise contacting the cell with an azide-modified sugar, as described herein. These methods also comprise contacting the cell with an active molecule conjugated to an azide reactive molecule, wherein the azide reactive molecule is chemically reactable with the azide of the azide-modified sugar. In these methods, the azide-modified sugar can be any of the azide-modified sugars described herein. In these methods, the azide-modified sugar can be acetylated as described herein.

In some embodiments, the azide reactive molecule is chosen from a cyclooctvne, a norbornene, an oxanorbornadiene, a phosphine, a dialkyl phosphine, a trialkyl phosphine, a phosphinothiol, a phosphinophenol, a cyclooctene, a tetrazine, a tetrazole, and a quadricyclane. In some embodiments, the azide reactive molecule is a cyclooctyne. In some embodiments, the azide reactive molecule is a norbornene. In some embodiments, the azide reactive molecule is an oxanorbornadiene. In some embodiments, the azide reactive molecule is a phosphine. In some embodiments, the azide reactive molecule is a dialkyl phosphine. In some embodiments, the azide reactive molecule is a trialkyl phosphine. In some embodiments, the azide reactive molecule is a phosphinothiol. In some embodiments, the azide reactive molecule is a phosphinophenol. In some embodiments, the azide reactive molecule is a cyclooctene. In some embodiments, the azide reactive molecule is a tetrazine. In some embodiments, the azide reactive molecule is a tetrazole. In some embodiments, the azide reactive molecule is a quadricyclane.

In some embodiments, the azide reactive molecule is chosen from dibenzocyclooctyne (DBCO), bicyclo[6.1.0]nonyne (BCN), methyltetrazine, and trans-cyclooctene (TCO). In some embodiments, the azide reactive molecule is DBCO. In some embodiments, the azide reactive molecule is BCN. In some embodiments, the azide reactive molecule is methyltetrazine. In some embodiments, the azide reactive molecule is TCO.

In some embodiments, the azide reactive molecule is chosen from a cyclooctyne, a cyclooctene, and a tetrazine. In some embodiments, the cyclooctyne is DBCO, BCN, monotluorinated cyclooctyne, difluorocyclooctyne, dimethoxyazacyclooctyne, dibenzoazacyclooctyne, biarylazacyclooctynone, 2,3,6,7-tetramethoxy-dibenzocyclooctyne, sulfonylated dibenzocyclooctyne, pyrrolocyclooctyne, or carboxymethylmonobenzocyclooctyne. In some embodiments, the cyclooctene is TCO. In some embodiments, the tetrazine is methyltetrazine, diphenyltetrazine, 3,6-di-(2-pyridyl)-s-tetrazine, 3,6-diphenyl-s-tetrazine, 3-(5-aminopyridin-2-yl)-6-(pyridin-2-yl)-s-tetrazine, or N-benzoyl-3-(5-aminopyridin-2-yl)-6-(pyridin-2-yl)-s-tetrazine.

In some embodiments, the active molecule is a polypeptide or peptide. In some embodiments, the active molecule is a mimotope of a polypeptide or peptide. The active molecule serves as an antigen for binding to an antibody, or antigen binding fragment thereof. In some embodiments, the antigen binding fragment is single-chain antibody (ScFv), a Fab fragment, or a F(ab′)₂ fragment.

In some embodiments, the active molecule is HER-2. In some embodiments, the active molecule is a HER-2 mimotope. In some embodiments, the HER-2 mimotope is a peptide comprising the amino acid sequence QLGPYELWELSH (SEQ ID NO:1) or LLGPYELWELSH (SEQ ID NO:2). In some embodiments, the HER-2 mimotope is a peptide comprising the amino acid sequence QLGPYELWELSH (SEQ ID NO:1). In some embodiments, the HER-2 mimotope is a peptide comprising the amino acid sequence LLGPYELWELSH (SEQ ID NO:2). In some embodiments, the HER-2 mimotope is a polypeptide comprising the formula SerGlyGlyGlySerGlyGlyGlyGlnLeuXaa¹ProTyrGluXaa² TrpGluLeuXaa³His (SEQ ID NO:3), wherein: a) Xaa¹ is Cys, Xaa² is Leu, and Xaa³ is Ser (SEQ ID NO:4), b) Xaa¹ is Gly, Xaa² is Cys, and Xaa³ is Ser (SEQ ID NO:5), c) Xaa¹ is Gly, Xaa² is Leu, and Xaa³ is Cys (SEQ ID NO:6), or d) Xaa¹ is Gly, Xaa² is Leu, and Xaa³ is Ser (SEQ ID NO:87). In some embodiments, Xaa¹ is Cys, Xaa² is Leu, and Xaa³ is Ser (SEQ ID NO:4). In some embodiments, Xaa¹ is Gly, Xaa² is Cys, and Xaa³ is Ser (SEQ ID NO:5). In some embodiments, Xaa¹ is Gly, Xaa² is Leu, and Xaa³ is Cys (SEQ ID NO:6). In some embodiments, Xaa¹ is Gly, Xaa² is Leu, and Xaa³ is Ser (SEQ ID NO:87). In some embodiments, the HER-2 mimotope is a polypeptide comprising the formula SerGlyGlyGly SerGlyGlyGlyGlnXaa¹LeuXaa²GlyXaa³ProXaa⁴TyrXaa⁵GluXaa⁶LeuXaa⁷TrpXaa⁸GluXaa⁹L euXaa¹⁰SerXaa¹¹His (SEQ ID NO:7), wherein: a) Xaa¹ is Cys and Xaa², Xaa³, Xaa⁴, Xaa⁵, Xaa⁶, Xaa⁷, Xaa⁸, Xaa⁹, Xaa¹⁰, and Xaa¹¹ are absent (SEQ ID NO:8); b) Xaa² is Cys and Xaa¹, Xaa³, Xaa⁴, Xaa⁵, Xaa⁶, Xaa⁷, Xaa⁸, Xaa⁹, Xaa¹⁰, and Xaa¹¹ are absent (SEQ ID NO:9); c) Xaa³ is Cys and Xaa¹, Xaa², Xaa⁴, Xaa⁵, Xaa⁶, Xaa⁷, Xaa⁸, Xaa⁹, Xaa¹⁰, and Xaa¹¹ are absent (SEQ ID NO:10); d) Xaa⁴ is Cys and Xaa¹, Xaa², Xaa³, Xaa⁵, Xaa⁶, Xaa⁷, Xaa⁸, Xaa⁹, Xaa¹⁰, and Xaa¹¹ are absent (SEQ ID NO:11); e) Xaa⁵ is Cys and Xaa¹, Xaa², Xaa³, Xaa⁴, Xaa⁶, Xaa⁷, Xaa⁸, Xaa⁹, Xaa¹⁰, and Xaa¹¹ are absent (SEQ ID NO:12); f) Xaa⁶ is Cys and Xaa¹, Xaa², Xaa³, Xaa⁴, Xaa⁵, Xaa⁷, Xaa⁸, Xaa⁹, Xaa¹⁰, and Xaa¹¹ are absent (SEQ ID NO:13); g) Xaa⁷ is Cys and Xaa¹, Xaa², Xaa³, Xaa⁴, Xaa⁵, Xaa⁶, Xaa⁸, Xaa⁹, Xaa¹⁰, and Xaa¹¹ are absent (SEQ ID NO:14); h) Xaa⁸ is Cys and Xaa¹, Xaa², Xaa³, Xaa⁴, Xaa⁵, Xaa⁶, Xaa⁷, Xaa⁹, Xaa¹⁰, and Xaa¹¹ are absent (SEQ ID NO:15); i) Xaa⁹ is Cys and Xaa¹, Xaa², Xaa³, Xaa⁴, Xaa⁵, Xaa⁶, Xaa⁷, Xaa⁸, Xaa¹⁰, and Xaa¹¹ are absent (SEQ ID NO:16); j) Xaa¹⁰ is Cys and Xaa¹, Xaa², Xaa³, Xaa⁴, Xaa⁵, Xaa⁶, Xaa⁷, Xaa⁸, Xaa⁹, and Xaa¹¹ are absent (SEQ ID NO:17); or k) Xaa¹¹ is Cys and Xaa¹, Xaa², Xaa³, Xaa⁴, Xaa⁵, Xaa⁶, Xaa⁷, Xaa⁸, Xaa⁹, and Xaa¹⁰ are absent (SEQ ID NO:18). In some embodiments, Xaa¹ is Cys and Xaa², Xaa³, Xaa⁴, Xaa⁵, Xaa⁶, Xaa⁷, Xaa⁸, Xaa⁹, Xaa¹⁰, and Xaa¹¹ are absent (SEQ ID NO:8). In some embodiments, Xaa² is Cys and Xaa¹, Xaa³, Xaa⁴, Xaa⁵, Xaa⁶, Xaa⁷, Xaa⁸, Xaa⁹, Xaa¹⁰, and Xaa¹¹ are absent (SEQ ID NO:9). In some embodiments, Xaa³ is Cys and Xaa¹, Xaa², Xaa⁴, Xaa⁵, Xaa⁶, Xaa⁷, Xaa⁸, Xaa⁹, Xaa¹⁰, and Xaa¹¹ are absent (SEQ ID NO:10). In some embodiments, Xaa⁴ is Cys and Xaa¹, Xaa², Xaa³, Xaa⁵, Xaa⁶, Xaa⁷, Xaa⁸, Xaa⁹, Xaa¹⁰, and Xaa¹¹ are absent (SEQ ID NO:11). In some embodiments, Xaa⁵ is Cys and Xaa¹, Xaa², Xaa³, Xaa⁴, Xaa⁶, Xaa⁷, Xaa⁸, Xaa⁹, Xaa¹⁰, and Xaa¹¹ are absent (SEQ ID NO:12). In some embodiments, Xaa⁶ is Cys and Xaa¹, Xaa², Xaa³, Xaa⁴, Xaa⁵, Xaa⁷, Xaa⁸, Xaa⁹, Xaa¹⁰, and Xaa¹¹ are absent (SEQ ID NO:13). In some embodiments, Xaa⁷ is Cys and Xaa¹, Xaa², Xaa³, Xaa⁴, Xaa⁵, Xaa⁶, Xaa⁸, Xaa⁹, Xaa¹⁰, and Xaa¹¹ are absent (SEQ ID NO:14). In some embodiments, Xaa⁸ is Cys and Xaa¹, Xaa², Xaa³, Xaa⁴, Xaa⁵, Xaa⁶, Xaa⁷, Xaa⁹, Xaa¹⁰, and Xaa¹¹ are absent (SEQ ID NO:15). In some embodiments, Xaa⁹ is Cys and Xaa¹, Xaa², Xaa³, Xaa⁴, Xaa⁵, Xaa⁶, Xaa⁷, Xaa⁸, Xaa¹⁰, and Xaa¹¹ are absent (SEQ ID NO:16). In some embodiments, Xaa¹⁰ is Cys and Xaa¹, Xaa², Xaa³, Xaa⁴, Xaa⁵, Xaa⁶, Xaa⁷, Xaa⁸, Xaa⁹, and Xaa¹¹ are absent (SEQ ID NO:17). In some embodiments, Xaa¹¹ is Cys and Xaa¹, Xaa², Xaa³, Xaa⁴, Xaa⁵, Xaa⁶, Xaa⁷, Xaa⁸, Xaa⁹, and Xaa¹⁰ are absent (SEQ ID NO:18). In some embodiments, when the active molecule is HER-2 or a HER-2 mimotope, the antibody is trastuzumab.

In some embodiments, the active molecule is the F glycoprotein of Respiratory Syncytial Virus (RSV). In some embodiments, the active molecule is an F glycoprotein mimotope. In some embodiments, the F glycoprotein mimotope is a peptide comprising the amino acid sequence NSELLSLINDMPITNDQKKLMSNN (SEQ ID NO:19). In some embodiments, when the active molecule is the F glycoprotein of RSV or an F glycoprotein mimotope, the antibody is palivizumab or motavizumab. In some embodiments, when the active molecule is the F glycoprotein of RSV or an F glycoprotein mimotope, the antibody is palivizumab. In some embodiments, when the active molecule is the F glycoprotein of RSV or an F glycoprotein mimotope, the antibody is motavizumab.

In some embodiments, the active molecule is Epidermal Growth Factor Receptor (EGFR). In some embodiments, the active molecule is an EGFR mimotope. In some embodiments, the EGFR mimotope is a peptide comprising the amino acid sequence IYPPLLRTSQAM (SEQ ID NO:20), AYPPYLRSMTLY (SEQ ID NO:21), YPPAERTYSTNY (SEQ ID NO:22), CPKWDAARC (SEQ ID NO:23), or CGPTRWRSC (SEQ ID NO:24). In some embodiments, the EGFR mimotope is a peptide comprising the amino acid sequence IYPPLLRTSQAM (SEQ ID NO:20). In some embodiments, the EGFR mimotope is a peptide comprising the amino acid sequence AYPPYLRSMTLY (SEQ ID NO:21). In some embodiments, the EGFR mimotope is a peptide comprising the amino acid sequence YPPAERTYSTNY (SEQ ID NO:22). In some embodiments, the EGFR mimotope is a peptide comprising the amino acid sequence CPKWDAARC (SEQ ID NO:23). In some embodiments, the EGFR mimotope is a peptide comprising the amino acid sequence CGPTRWRSC (SEQ ID NO:24). In some embodiments, when the active molecule is EGFR or an EGFR mimotope, the antibody is panitumumab.

In some embodiments, the active molecule is the Vi antigen of Salmonella enterica. In some embodiments, the active molecule is a Vi antigen mimotope. In some embodiments, the Vi antigen mimotope is a peptide comprising the amino acid sequence TSHHDSHGLHRV (SEQ ID NO:25), TSHHDSHGDHHV (SEQ ID NO:26), TSHHDSHGVHRV (SEQ ID NO:27), TSHHDSHDLHRV (SEQ ID NO:28), TSHHDYHGLHRV (SEQ ID NO:29), ENHSPVNIAHKL (SEQ ID NO:30), ENHSPVNIAHKV (SEQ ID NO:31), ENHSPVNIDHKL (SEQ ID NO:32), EDHSPVNIDHKL (SEQ ID NO:33), ENHYPLHAAHRI (SEQ ID NO:34), ESHQHVHDLVFL (SEQ ID NO:35), PGHHDFVGLHHL (SEQ ID NO:36), ENHYPVNIAHKL (SEQ ID NO:37), or DNHSPVNIAHKL (SEQ ID NO:38). In some embodiments, the Vi antigen mimotope is a peptide comprising the amino acid sequence TSHHDSHGLHRV (SEQ ID NO:25). In some embodiments, the Vi antigen mimotope is a peptide comprising the amino acid sequence TSHHDSHGDHHV (SEQ ID NO:26). In some embodiments, the Vi antigen mimotope is a peptide comprising the amino acid sequence TSHHDSHGVHRV (SEQ ID NO:27). In some embodiments, the Vi antigen mimotope is a peptide comprising the amino acid sequence TSHHDSHDLHRV (SEQ ID NO:28). In some embodiments, the Vi antigen mimotope is a peptide comprising the amino acid sequence TSHHDYHGLHRV (SEQ ID NO:29). In some embodiments, the Vi antigen mimotope is a peptide comprising the amino acid sequence ENHSPVNIAHKL (SEQ ID NO:30). In some embodiments, the Vi antigen mimotope is a peptide comprising the amino acid sequence ENHSPVNIAHKV (SEQ ID NO:31). In some embodiments, the Vi antigen mimotope is a peptide comprising the amino acid sequence ENHSPVNIDHKL (SEQ ID NO:32). In some embodiments, the Vi antigen mimotope is a peptide comprising the amino acid sequence EDHSPVNIDHKL (SEQ ID NO:33). In some embodiments, the Vi antigen mimotope is a peptide comprising the amino acid sequence ENHYPLHAAHRI (SEQ ID NO:34). In some embodiments, the Vi antigen mimotope is a peptide comprising the amino acid sequence ESHQHVHDLVFL (SEQ ID NO:35). In some embodiments, the Vi antigen mimotope is a peptide comprising the amino acid sequence PGHHDFVGLHHL (SEQ ID NO:36). In some embodiments, the Vi antigen mimotope is a peptide comprising the amino acid sequence ENHYPVNIAHKL (SEQ ID NO:37). In some embodiments, the Vi antigen mimotope is a peptide comprising the amino acid sequence DNHSPVNIAHKL (SEQ ID NO:38). In some embodiments, when the active molecule is the Vi antigen of Salmonella enterica or a Vi antigen mimotope, the antibody is ATVi.

In some embodiments, the active molecule is a peptide of H5N1 Influenza Virus. In some embodiments, the peptide of H5N1 Influenza Virus comprises the amino acid sequence YINPHMYWMSVA (SEQ ID NO:39), HTPPPQPYRTHI (SEQ ID NO:40), TFWVQTAKPNPL (SEQ ID NO:41), GHPSKTSGHPLT (SEQ ID NO:42), TYVNIVLYDDVE (SEQ ID NO:43), TTNFLNHAIAHK (SEQ ID NO:44), YYNPSPPNPRTQ (SEQ ID NO:45), TESPQYIALSFH (SEQ ID NO:46), HWYDWLTRYSHL (SEQ ID NO:47), ATYTTDAQSYHM (SEQ ID NO:48), DHYWHRSNTLSH (SEQ ID NO:49), VTSHDLKKSGTW (SEQ ID NO:50), WEFAYKNTRYYW (SEQ ID NO:51), SWTSLPLHEAIH (SEQ ID NO:52), TLAHTHTSTSSF (SEQ ID NO:53), WHWSFFASPLPA (SEQ ID NO:54), WHWNARNWSSQQ (SEQ ID NO:55), CWTSLPLHEAIH (SEQ ID NO:56), VPTECSGRTSCT (SEQ ID NO:57), WSNHWWHSKWAI (SEQ ID NO:58), HIWNWSNWTQWT (SEQ ID NO:59), HIFHNTHWWQRW (SEQ ID NO:60), TNYDYIPDTQNT (SEQ ID NO:61), SWSSHSNSTPTSYNTNQTQNPTSTSTNQPNNN (SEQ ID NO:62), or NHEKIPKSSWSSHWKYNTNQEDNKTIKPNDNEYKVK (SEQ ID NO:63). In some embodiments, the peptide of H5N1 Influenza Virus comprises the amino acid sequence YINPHMYWMSVA (SEQ ID NO:39). In some embodiments, the peptide of H5N1 Influenza Virus comprises the amino acid sequence HTPPPQPYRTHI (SEQ ID NO:40). In some embodiments, the peptide of H5N1 Influenza Virus comprises the amino acid sequence TFWVQTAKPNPL (SEQ ID NO:41). In some embodiments, the peptide of H5N1 Influenza Virus comprises the amino acid sequence GHPSKTSGHPLT (SEQ ID NO:42). In some embodiments, the peptide of H5N1 Influenza Virus comprises the amino acid sequence TYVNIVLYDDVE (SEQ ID NO:43). In some embodiments, the peptide of H5N1 Influenza Virus comprises the amino acid sequence TTNFLNHAIAHK (SEQ ID NO:44). In some embodiments, the peptide of H5N1 Influenza Virus comprises the amino acid sequence YYNPSPPNPRTQ (SEQ ID NO:45). In some embodiments, the peptide of H5N1 Influenza Virus comprises the amino acid sequence TESPQYIALSFH (SEQ ID NO:46). In some embodiments, the peptide of H5N1 Influenza Virus comprises the amino acid sequence HWYDWLTRYSHL (SEQ ID NO:47). In some embodiments, the peptide of H5N1 Influenza Virus comprises the amino acid sequence ATYTTDAQSYHM (SEQ ID NO:48). In some embodiments, the peptide of H5N1 Influenza Virus comprises the amino acid sequence DHYWHRSNTLSH (SEQ ID NO:49). In some embodiments, the peptide of H5N1 Influenza Virus comprises the amino acid sequence VTSHDLKKSGTW (SEQ ID NO:50). In some embodiments, the peptide of H5N1 Influenza Virus comprises the amino acid sequence WEFAYKNTRYYW (SEQ ID NO:51). In some embodiments, the peptide of H5N1 Influenza Virus comprises the amino acid sequence SWTSLPLHEAIH (SEQ ID NO:52). In some embodiments, the peptide of H5N1 Influenza Virus comprises the amino acid sequence TLAHTHTSTSSF (SEQ ID NO:53). In some embodiments, the peptide of H5N1 Influenza Virus comprises the amino acid sequence WHWSFFASPLPA (SEQ ID NO:54). In some embodiments, the peptide of H5N1 Influenza Virus comprises the amino acid sequence WHWNARNWSSQQ (SEQ ID NO:55). In some embodiments, the peptide of H5N1 Influenza Virus comprises the amino acid sequence CWTSLPLHEAIH (SEQ ID NO:56). In some embodiments, the peptide of H5N1 Influenza Virus comprises the amino acid sequence VPTECSGRTSCT (SEQ ID NO:57). In some embodiments, the peptide of H5N1 Influenza Virus comprises the amino acid sequence WSNHWWHSKWAI (SEQ ID NO:58). In some embodiments, the peptide of H5N1 Influenza Virus comprises the amino acid sequence HIWNWSNWTQWT (SEQ ID NO:59). In some embodiments, the peptide of H5N1 Influenza Virus comprises the amino acid sequence HIFHNTHWWQRW (SEQ ID NO:60). In some embodiments, the peptide of H5N1 Influenza Virus comprises the amino acid sequence TNYDYIPDTQNT (SEQ ID NO:61). In some embodiments, the peptide of H5N1 Influenza Virus comprises the amino acid sequence SWSSHSNSTPTSYNTNQTQNP TSTSTNQPNNN (SEQ ID NO:62). In some embodiments, the peptide of H5N1 Influenza Virus comprises the amino acid sequence NHIEKIPKSSWSSHWKYNTNQEDNKTIKPNDN EYKVK (SEQ ID NO:63). In some embodiments, when the active molecule is peptide of the H5N1 Influenza Virus, the antibody is AVFluIgG01.

In some embodiments, the active molecule is CD147. In some embodiments, the CD147 comprises the amino acid sequence YPHFHKHTLRGH (SEQ ID NO:64), YPHFHKHSLRGQ (SEQ ID NO:65), DHKPFKPTHRTL (SEQ ID NO:66), FHKPFKPTHRTL (SEQ ID NO:67), QSSCHKHSVRGR (SEQ ID NO:68), QSSFSNHSVRRR (SEQ ID NO:69), or DFDVSFLSARMR (SEQ ID NO:70). In some embodiments, the CD147 comprises the amino acid sequence YPHFHKHTLRGH (SEQ ID NO:64). In some embodiments, the CD147 comprises the amino acid sequence YPHFHKHSLRGQ (SEQ ID NO:65). In some embodiments, the CD147 comprises the amino acid sequence DHKPFKPTHRTL (SEQ ID NO:66). In some embodiments, the CD147 comprises the amino acid sequence FHKPFKPTHRTL (SEQ ID NO:67). In some embodiments, the CD147 comprises the amino acid sequence QSSCHKHSVRGR (SEQ ID NO:68). In some embodiments, the CD147 comprises the amino acid sequence QSSFSNHSVRRR (SEQ ID NO:69). In some embodiments, the CD147 comprises the amino acid sequence DFDVSFLSARMR (SEQ ID NO:70). In some embodiments, when the active molecule is CD147, the antibody is metuximab.

In some embodiments, the active molecule is a protein on Schistosoma mansoni. In some embodiments, the protein on Schistosoma mansoni comprises the amino acid sequence VLLRRIGG (SEQ ID NO:71), HLLRLSEI (SEQ ID NO:72), SLLTYMKM (SEQ ID NO:73), or YLLQKLRN (SEQ ID NO:74). In some embodiments, the protein on Schistosoma mansoni comprises the amino acid sequence VLLRRIGG (SEQ ID NO:71). In some embodiments, the protein on Schistosoma mansoni comprises the amino acid sequence HLLRLSEI (SEQ ID NO:72). In some embodiments, the protein on Schistosoma mansoni comprises the amino acid sequence SLLTYMKM (SEQ ID NO:73). In some embodiments, the protein on Schistosoma mansoni comprises the amino acid sequence YLLQKLRN (SEQ ID NO:74). In some embodiments, when the active molecule is a protein on Schistosoma mansoni, the antibody is 152-66-9b.

Additional antibody, or antigen-binding fragment thereof, and active molecule pairs are suitable. Such pairs include, but are not limited to, adalimumab and TNF, alemtuzumab and CD52, alirocumab and PCSK9, atezolizumab and PD-L1, avelumab and PD-L1, belimumab and BLyS, bevacizumab and VEGF, blinatumomab and CD19/DC3 (bi-specific), brodalumab and IL-17RA, burosumab and FGF23, canakinumab and IL-1, certolizumab and TNFα, cetuximab and EGFR, daratumumab and CD38, denosumab and RANKL, dinituximab and GD2, dupilumab and IL-4Ra, durvalumab and PD-L1, elotuzumab and SLAMF7, emicizumab and Factor-IXa/Factor X (bi-specific), erenumab and calcitonin gene-related peptide receptor, evolocumab and PCSK9, gemtuzumab and CD33, golimumab and TNF, guselkumab and IL-23, ibalizumab and CD4, ibritumomab and CD20, infliximab and TNFα, inotuzumab and CD22, ipilimumab and CTLA-4, ixekizumab and IL-17A, mepolizumab and IL-5, natalizumab and integrin receptor, necitumumab and EGFR, nivolumab and PD-1, obinuntuzumab and CD20, ocrelizumab and CD20, ofatumumab and CD20, olaratumab and PDGFR-α, omalizumab and IgE antibody, pembrolizumab and PD-1, pertuzumab and HER2/neu, ramucirumab and VEGFR2, ranibizumab and VEGF, reslizumab and IL-5, rituximab and CD20, sarilumab and IL-6R, secukinumab and IL-17A, siltuximab and IL-6, tocilizumab and IL-6R, ustekinumab and IL-12/IL-23, and vendolizumab and integrin receptor.

The present disclosure also provides methods of anchoring at least two active molecules on the surface of a cell. In these methods, the cell is contacted with an azide-modified sugar. In these methods, the cell is contacted with a first active molecule conjugated to a first azide reactive molecule, wherein the first azide reactive molecule is chemically reactable with the azide of the azide-modified sugar. In these methods, the cell is also contacted with a second active molecule conjugated to a second azide reactive molecule, wherein the second azide reactive molecule is chemically reactable with the azide of the azide-modified sugar. In these methods, the azide-modified sugar can be any of the azide-modified sugars described herein. In these methods, the first azide reactive molecule and second azide reactive molecule are, independently, any of the azide reactive molecules described herein. In some embodiments, the first azide reactive molecule and second azide reactive molecule are different azide reactive molecules. In some embodiments, the first azide reactive molecule and second azide reactive molecule are the same azide reactive molecule. In these methods, the first active molecule and second active molecule are, independently, a polypeptide or peptide, or mimotope thereof, that serve as antigens for binding to a bi-specific antibody, or antigen binding fragment thereof. In these methods, the antigen binding fragment can be any of the antigen binding fragments described herein. In some embodiments, the first active molecule and second active molecule are, independently, a mimotope for HER-2, the F glycoprotein of RSV, EGFR, the Vi antigen of Salmonella enterica, a peptide of H5N1 Influenza Virus, CD147, or a protein on Schistosoma mansoni. In some embodiments, the antibody, or antigen-binding fragment thereof, and active molecule pairs are any of those described herein.

The present disclosure also provides methods of anchoring an active molecule on the surface of a cell. In these methods, the cell is contacted with an azide-modified sugar. In these methods, the cell is also contacted with a first small interactive peptide conjugated to an azide reactive molecule, wherein the azide reactive molecule is chemically reactable with the azide of the azide-modified sugar. In these methods, the cell is also contacted with a second small interactive peptide conjugated to an active molecule, wherein the first small interactive peptide interacts with the second small interactive peptide. In these methods, the azide-modified sugar can be any of the azide-modified sugars described herein. In these methods, the azide reactive molecule can be any of the azide reactive molecules described herein. In these methods, the active molecule can be any of the polypeptides or peptides, or mimotopes thereof, described herein that serve as antigens for binding to an antibody, or antigen binding fragment thereof. In some embodiments, the active molecule is a mimotope for HER-2, the F glycoprotein of RSV, EGFR, the Vi antigen of Salmonella enterica, a polypeptide on H5N1 Influenza Virus, CD147, or a protein on Schistosoma mansoni. In these methods, the antigen binding fragment can be any of the antigen binding fragments described herein. In these methods, the antibody, or antigen-binding fragment thereof, and active molecule pairs can be any of those described herein.

In some embodiments, the first small interactive peptide and the second small interactive peptide are leucine zipper pairs. Suitable examples of small interactive peptides are the c-jun and c-fos zipper domains, which generally are polypeptides of less than 50 amino acid residues, including helix-initiating and helix-terminating segments. While c-jun can form homodimers, c-fos cannot; and c-fos:c-jun heterodimers are significantly more stable than c-jun:c-jun homodimers. In some embodiments, the first small interactive peptide and the second small interactive peptide are chosen from jun/fos, mad/max, myc/max, and NZ/CZ zipper domains. In some embodiments, the first small interactive peptide and the second small interactive peptide are jun and fos zipper domains. In some embodiments, the first small interactive peptide and the second small interactive peptide are mad/max zipper domains. In some embodiments, the first small interactive peptide and the second small interactive peptide are chosen from myc/max zipper domains. In some embodiments, the first small interactive peptide and the second small interactive peptide are NZ/CZ zipper domains. In some embodiments, the small interactive protein peptides are antiparallel zippers, such as that from Thermus thermophilus seryl-tRNA synthetase.

In some embodiments, one of the first small interactive peptide and the second small interactive peptide is a c-jun polypeptide comprising an amino acid sequence chosen from CSGGASLERIARLEEKVKTLKAQNSELASTANMLREQVAQLKQKGAP (SEQ ID NO:75), SGASLERIARLEEKVKTLKAQNSELASTANMLREQVAQLKQKGAPSG GC (SEQ ID NO:76), CSGGASLERIARLEEKVKSFKAQNSENASTANMLREQVAQL KQKGAP (SEQ ID NO:77), SGASLERIARLEEKVKSFKAQNSENASTANMLREQVA QLKQKGAPSGGC (SEQ ID NO:78), CSGASLERIARLEEKVKSFKAQNSENASTANM LREQVAQLKQKGAP (SEQ ID NO:79), and GASLERIARLEEKVKTLKAQNSELAS TANMLREQVAQLKQKGAPSGGC (SEQ ID NO:80), and the other of the first small interactive peptide and the second small interactive peptide is a c-fos polypeptide comprising an amino acid sequence chosen from ASRELTDTLQAETDQLEDEKSALQTEIANLLKEK EKLEGAP (SEQ ID NO:81), ASRETDTLQAETDQLEDEKSALQTEIANLLKEKEKL EGAP (SEQ ID NO:82), and SGASRELTDTLQAETDQLEDEKSALQTEIANLLKEK EKLEGAP (SEQ ID NO:83). In some embodiments, the c-jun polypeptide comprises the amino acid sequence CSGGASLERIARLEEKVKTLKAQNSELASTANMLREQVAQLKQ KGAP (SEQ ID NO:75). In some embodiments, the c-jun polypeptide comprises the amino acid sequence SGASLERIARLEEKVKTLKAQNSELASTANMLREQVAQLKQKG APSGGC (SEQ ID NO:76). In some embodiments, the c-jun polypeptide comprises the amino acid sequence CSGGASLERIARLEEKVKSFKAQNSENASTANMLREQVAQLKQ KGAP (SEQ ID NO:77). In some embodiments, the c-jun polypeptide comprises the amino acid sequence SGASLERIARLEEKVKSFKAQNSENASTANMLREQVAQLKQK GAPSGGC (SEQ ID NO:78). In some embodiments, the c-jun polypeptide comprises the amino acid sequence CSGASLERIARLEEKVKSFKAQNSENASTANMLREQVAQLKQK GAP (SEQ ID NO:79). In some embodiments, the c-jun polypeptide comprises the amino acid sequence GASLERIARLEEKVKTLKAQNSELASTANMLREQVAQLKQKGA PSGGC (SEQ ID NO:80). In some embodiments, the c-fos polypeptide comprises the amino acid sequence ASRELTDTLQAETDQLEDEKSALQTEIANLLKEKEKLEGAP (SEQ ID NO:81). In some embodiments, the c-fos polypeptide comprises the amino acid sequence ASRETDTLQAETDQLEDEKSALQTEIANLLKEKEKLEGAP (SEQ ID NO:82). In some embodiments, the c-fos polypeptide comprises the amino acid sequence SGASRELTDTLQAETDQLEDEKSALQTEIANLLKEKEKLEGAP (SEQ ID NO:83).

Additional extended serine-glycine linkers can be inserted between the c-fos sequence and the sequence to which it is linked. Additional extended serine-glycine linkers can be inserted between the c-jun sequence and the sequence to which it is linked.

In some embodiments, one of the first small interactive peptide and the second small interactive peptide is an NZ domain comprising an amino acid sequence chosen from ALKK ELQANKKELAQLKWELQALKKELAQ (SEQ ID NO:84) and SGGGSGASALKKELQ ANKKELAQLKWELQALKKELAQGAPGS (SEQ ID NO:85), and the other of the first small interactive peptide and the second small interactive peptide is a CZ domain comprising an amino acid sequence is EQLEKKLQALEKKLAQLEWKNQALEKKLAQ (SEQ ID NO:86). In some embodiments, the NZ domain comprises the amino acid sequence ALKKELQANKKELAQLKWELQALKKELAQ (SEQ ID NO:84). In some embodiments, the NZ domain comprises the amino acid sequence SGGGSGASALKKELQ ANKKELAQLKWELQALKKELAQGAPGS (SEQ ID NO:85).

In any of the embodiments described herein, chemical linkers can be incorporated into the compounds. The chemical linkers can be included between any of the portions of the compounds. The chemical linkers can aid in facilitating spatial separation of the portions, increasing flexibility of the portions relative to each other, improving physical or functional characteristics (such as solubility, hydrophobicity, charge, cell-permeability, toxicity, biodistribution, or stability), or any combination of the above. Examples of chemical linkers include, but are not limited to, chains of one or more of the following: alkyl groups, alkenyl groups, amides, esters, thioesters, ketones, ethers, thioethers, disulfides, ethylene glycol, cycloalkyl groups, benzyl groups, heterocyclic groups, maleimidyl groups, hydrazones, urethanes, azoles, imines, haloalkyl groups, and carbamates, or any combination thereof. In some embodiments, the linkage between two amino acid sequences can comprise a linker. In some embodiments, the linker is a Ser/Gly linker, a Poly-Asparagine linker, or a linker comprising the amino acid sequence AGSSAAGSGS (SEQ ID NO:90). In some embodiments, the Poly-Asparagine linker comprises from about 8 to about 16 asparagine residues. In some embodiments, the Ser/Gly linker comprises GGSGGGSGGGSGGGSGGG (SEQ ID NO:91), GGSGGGSGGGSGGGSGGGSGGG (SEQ ID NO:92), GGSGG GSGGGSGGGSGGGSGGGSGGG (SEQ ID NO:93), SGGGGSGGGGSGGGG (SEQ ID NO:94), SGGGGSGGGGSGGGGSGGGG (SEQ ID NO:95), SGGGGSGGGGSG GGGSGGGGSGGGG (SEQ ID NO:96), SGGGS (SEQ ID NO:97), SGSG (SEQ ID NO:98), SGGGGS (SEQ ID NO:99), or SGSGG (SEQ ID NO:100).

### Methods of Employing CAR-T Cells

The present disclosure also provides methods of enhancing an immune response to a target cell in a human. In these methods, the target cell is contacted with an azide-modified sugar. In some embodiments, the target cell is in a human. In these methods, a CAR-T cell is introduced to the environment comprising the target cell. In some embodiments, the CAR-T cell is introduced into the human comprising the target cell. In these methods, the CAR-T cell comprises an extracellular FK506-binding protein (FKBP) domain or FRB domain functionally linked to a cytoplasmic signaling domain of the CART-T cell. In these methods, the target cell is contacted with a bifunctional FKBP or FRB domain binding compound, wherein a first portion of the bifunctional FKBP domain binding compound interacts with the FKBP domain on the CAR-T cell, and a second portion of the bifunctional FKBP or FRB domain binding compound interacts with the azide of the azide-modified sugar on the target cell. In these methods, the azide-modified sugar can be any of the azide-modified sugars described herein. In these methods, the azide-modified sugar can be acetylated as described herein.

Any CAR-T cell can be used in the present methods (see, Wilkins et al., Human Gene Ther. Meth., 2017, 28, 61-66). In some embodiments, the CAR-T cell comprises an extracellular FKBP domain or FRB domain functionally linked to the cytoplasmic signaling domain of the CART-T cell. In some embodiments, the CAR-T cell expresses a modified FKBP domain on the cell surface as a fusion with third-generation transmembrane/cytoplasmic signaling and activation domains.

In some embodiments, the CAR-T cell comprises an extracellular FKBP domain functionally linked to the cytoplasmic signaling domain of the CART-T cell. In some embodiments, the FKBP domain is a mutant FKBP domain. In some embodiments, the mutant FKBP domain is the F36V FKBP mutant domain comprising the amino acid sequence GVQVETISPGDGRTFPKRGQTCVVHYTGMLEDGKKVDSSRDRNKPFKFMLGKQEVI RGWEEGVAQMSVGQRAKLTISPDYAYGATGHPGIIPPHATLVFDVELLKLE (SEQ ID NO:88) or MGVQVETISPGDGRTFPKRGQTCVVHYTGMLEDGKKVDSSRDRNKPFK FMLGKQEVIRGWEEGVAQMSVGQRAKLTISPDYAYGATGHPGIIPPHATLVFDVELL KLE (SEQ ID NO:89). In some embodiments, the mutant FKBP domain comprises a C22S, C22A, or C22V substitution. In some embodiments, the CAR-T cell comprises an extracellular FRB domain functionally linked to the cytoplasmic signaling domain of the CART-T cell. In some embodiments, the FRB domain comprises a C61S, C61A, or C61V substitution.

In these methods, the bifunctional FKBP or FRB domain binding compound comprises a first portion which interacts with the FKBP domain on the CAR-T cell, and also comprises a second portion which interacts with the azide of the azide-modified sugar on the target cell. In some embodiments, the biftmnctional FKBP or FRB domain binding compound comprises the formula:

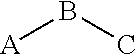

wherein: A is a small molecule ligand that binds to an FKBP domain or FRB domain; B is a chemical linker chosen from an alkyl, an alkenyl, an amide, an ester, a thioester, a ketone, an ether, a thioether, a disulfide, an ethylene glycol unit, a cycloalkyl, a benzyl, a heterocyclic, a maleimidyl, a hydrazone, a urethane, an azole, an imine, a haloalkyl, or a carbamate, or any combination thereof; and C is an azide reactive molecule such as any of those described herein.

In some embodiments, the small molecule ligand (A) comprises

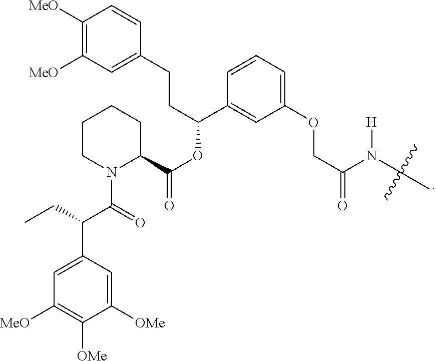 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

In any of the embodiments described herein, B is a chemical linker chosen from an alkyl, an alkenyl, an amide, an ester, a thioester, a ketone, an ether, a thioether, a disulfide, an ethylene glycol unit, a cycloalkyl, a benzyl, a heterocyclic, a maleimidyl, a hydrazone, a urethane, an azole, an imine, a haloalkyl, or a carbamate, or any combination thereof. In some embodiments, the chemical linker is an alkyl. In some embodiments, the chemical linker is an alkenyl. In some embodiments, the chemical linker is an amide. In some embodiments, the chemical linker is an ester. In some embodiments, the chemical linker is a thioester. In some embodiments, the chemical linker is a disulfide. In some embodiments, the chemical linker is a ketone. In some embodiments, the chemical linker is an ether. In some embodiments, the chemical linker is a thioether. In some embodiments, the chemical linker is an ethylene glycol unit. In some embodiments, the chemical linker is a cycloalkyl. In some embodiments, the chemical linker is a benzyl. In some embodiments, the chemical linker is a heterocyclic. In some embodiments, the chemical linker is a maleimidyl. In some embodiments, the chemical linker is a hydrazone. In some embodiments, the chemical linker is a urethane. In some embodiments, the chemical linker is an azole. In some embodiments, the chemical linker is an imine. In some embodiments, the chemical linker is a haloalkyl. In some embodiments, the chemical linker is a carbamate.

In some embodiments, the chemical linker is an alkyl or an ethylene glycol unit. In some embodiments, the chemical linker is an alkyl. In some embodiments, the chemical linker is a C₂-C₁₆alkyl. In some embodiments, the chemical linker is a C₄-C₂alkyl or a C₄-C₁₆alkyl. In some embodiments, the chemical linker is a C₄-C₁₀alkyl. In some embodiments, the chemical linker is C₄alkyl or C₁₀alkyl. In some embodiments, the chemical linker is an ethylene glycol unit. In some embodiments, the chemical linker is a polyethylene glycol (PEG). In some embodiments, the PEG is PEG2 to PEG16. In some embodiments, the PEG is PEG2, PEG3, or PEG4.

In some embodiments, the azide reactive molecule (C) is chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, and a quadricyclane. In some embodiments, the azide reactive molecule is a cyclooctyne. In some embodiments, the azide reactive molecule is a norbornene. In some embodiments, the azide reactive molecule is an oxanorbornadiene. In some embodiments, the azide reactive molecule is a phosphine. In some embodiments, the azide reactive molecule is a dialkyl phosphine. In some embodiments, the azide reactive molecule is a trialkyl phosphine. In some embodiments, the azide reactive molecule is a phosphinothiol. In some embodiments, the azide reactive molecule is a phosphinophenol. In some embodiments, the azide reactive molecule is a cyclooctene. In some embodiments, the azide reactive molecule is a tetrazine. In some embodiments, the azide reactive molecule is a tetrazole. In some embodiments, the azide reactive molecule is a quadricyclane.

In some embodiments, the azide reactive molecule is chosen from dibenzocyclooctyne (DBCO), bicyclo[6.1.0]nonyne (BCN), methyltetrazine, and trans-cyclooctene (TCO). In some embodiments, the azide reactive molecule is DBCO. In some embodiments, the azide reactive molecule is BCN. In some embodiments, the azide reactive molecule is methyltetrazine. In some embodiments, the azide reactive molecule is TCO.

In some embodiments, the azide reactive molecule is chosen from a cyclooctyne, a cyclooctene, and a tetrazine. In some embodiments, the cyclooctyne is DBCO, BCN, monotluorinated cyclooctyne, difluorocyclooctyne, dimethoxyazacyclooctyne, dibenzoazacyclooctyne, biarylazacyclooctynone, 2,3,6,7-tetramethoxy-dibenzocyclooctyne, sulfonylated dibenzocyclooctyne, pyrrolocyclooctyne, or carboxymethylmonobenzocyclooctyne. In some embodiments, the cyclooctene is TCO. In some embodiments, the tetrazine is methyltetrazine, diphenyltetrazine, 3,6-di-(2-pyridyl)-s-tetrazine, 3,6-diphenyl-s-tetrazine, 3-(5-aminopyridin-2-yl)-6-(pyridin-²-yl)-s-tetrazine, or N-benzoyl-3-(5-aminopyridin-2-yl)-6-(pyridin-2-yl)-s-tetrazine.

In some embodiments, the chemical linker is an alkyl group or an ethylene glycol unit, and the azide reactive molecule is chosen from a cyclooctyne, a cyclooctene, and a tetrazine. In some embodiments, the chemical linker is a C₂-C₁₆alkyl group, or a polyethylene glycol unit which is PEG2 to PEG16, and the azide reactive molecule is DBCO, BCN, TCO, or methyltetrazine. In some embodiments, the chemical linker is a C₄-C₁₀alkyl group or a polyethylene glycol unit which is PEG2, PEG3, or PEG4, and the azide reactive molecule is DBCO, BCN, TCO, or methyltetrazine. In some embodiments, the chemical linker is C₄alkyl group, C₁₀alkyl group, or PEG3, and the azide reactive molecule is DBCO or BCN In some embodiments, the small molecule ligand is

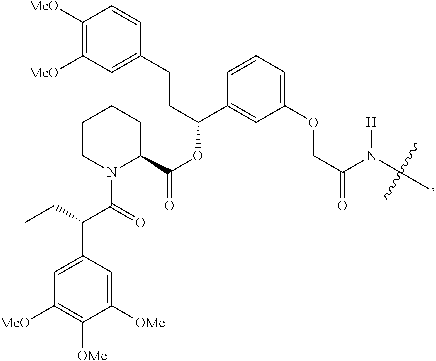 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

the chemical linker is C₄alkyl, C₁₀alkyl, or PEG3, and the azide reactive molecule is DBCO or BCN.

In some embodiments, the bifunctional FKBP or FRB domain binding compound comprises the formula:

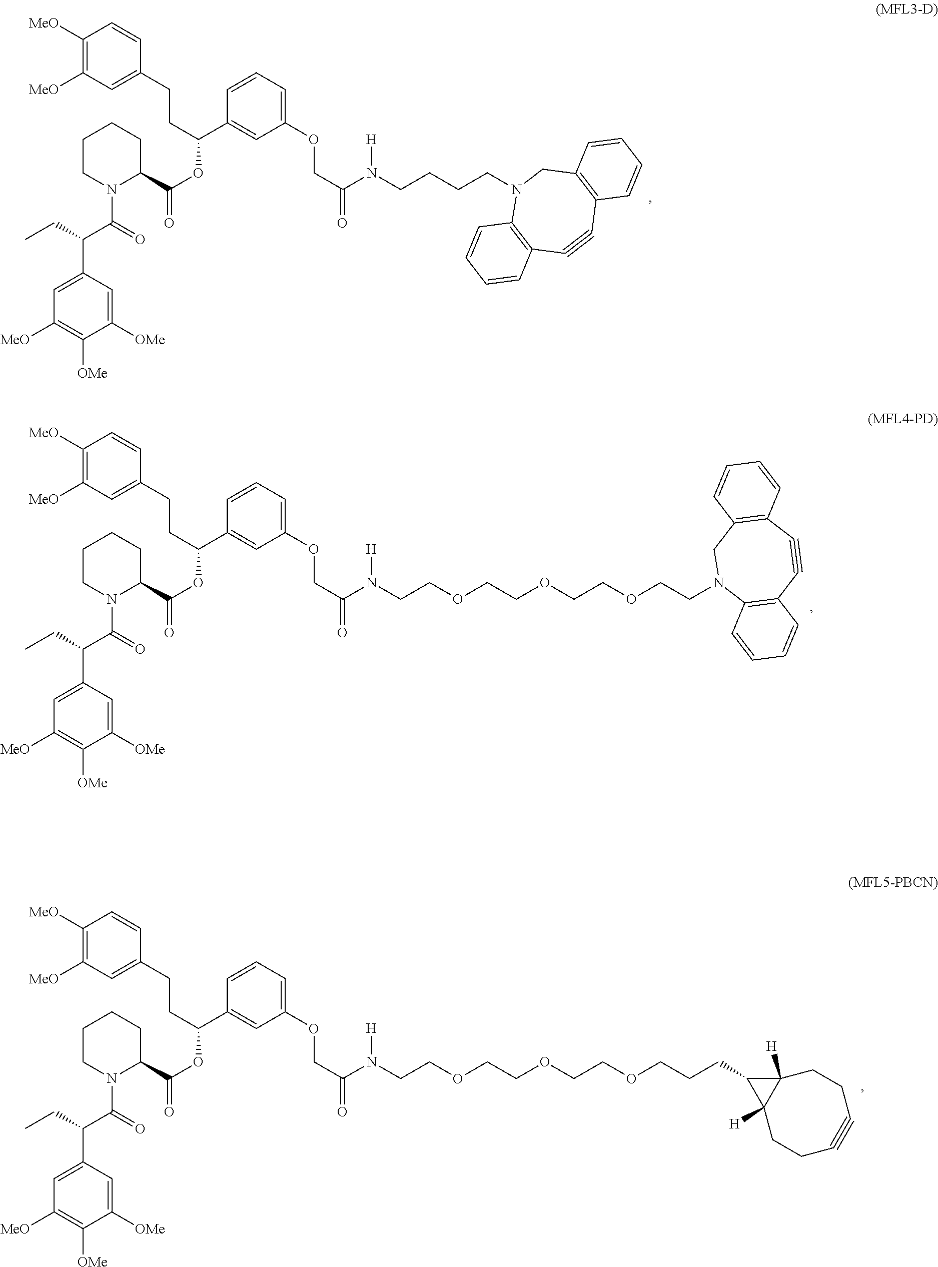 [SMILES: CC[C@H](C(=O)N1CCCC[C@H]1C(=O)O[C@H](CCc1ccc(OC)c(OC)c1)c1cccc(OCC(=O)NCCCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCC[C@@H]2[C@@H]3CCC#CCC[C@@H]32)c1)c1cc(OC)c(OC)c(OC)c1]

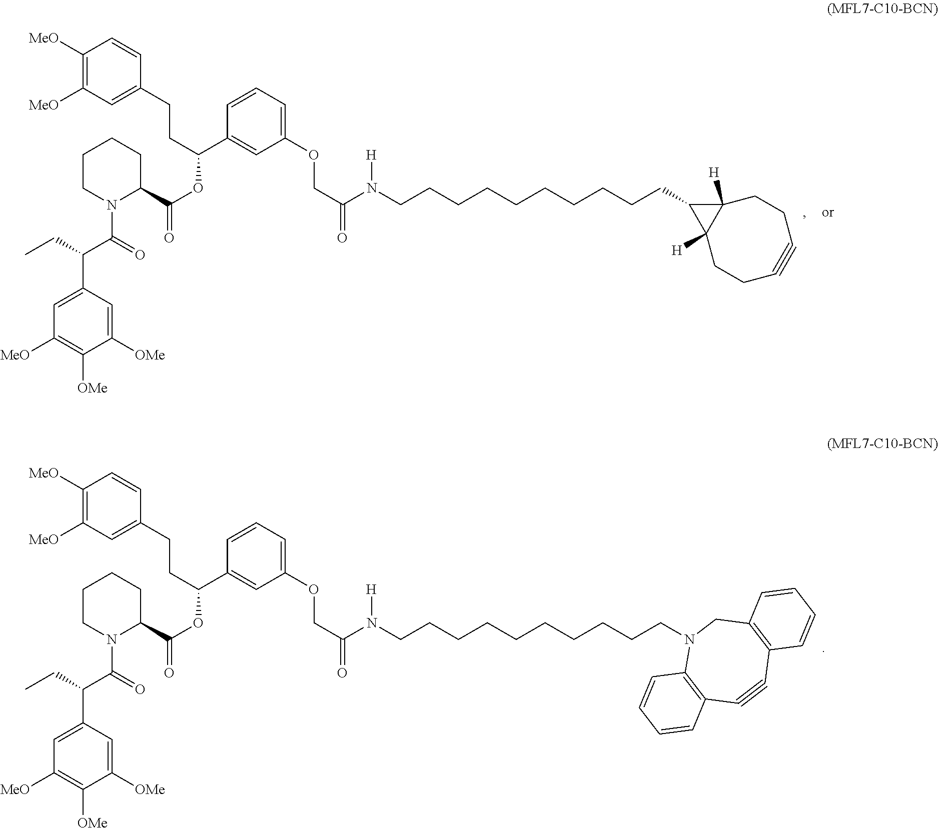 [SMILES: CC[C@H](C(=O)N1CCCC[C@H]1C(=O)O[C@H](CCc1ccc(OC)c(OC)c1)c1cccc(OCC(=O)NCCCCCCCCCC[C@@H]2[C@@H]3CCC#CCC[C@@H]32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCCCCCCCCCN2Cc3ccccc3C#Cc3ccccc32)c1)c1cc(OC)c(OC)c(OC)c1]

In some embodiments, the CAR-T cell comprising the extracellular FKBP domain or FRB domain functionally linked to the cytoplasmic signaling domain of the CART-T cell is pre-incubated with excess bifunctional compound prior to their contacting the target cells.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Labeling Cells with Azide-Modified Sugars

Demonstration of the presence of azides on a cell surface was achieved by treating a cell preparation with a fluorescent azide reactive molecule that reacts only with the azide group, without chemical reactivity with normal biological molecules. Initially, cells were cultured in a suitable culture vessel such that their level of confluency at the time of addition of the azide-modified sugar, AzNAM, was not more than 80%.

In particular, HeLa cells were plated in 6-well plates (2.5×10⁴ cells/well) and incubated for 48 hours in DMEM-10% FBS medium in a standard 5% CO₂ atmosphere. Medium from each well was removed, and each well was washed with 2 ml of phosphate buffered saline (PBS), and fresh DMEM-FBS (1 ml) added. Subsequently, varying amounts of AzNAM (solubilized in DMSO) were added to the wells in small (10 μl) volumes to produce the desired final concentrations in the range 10-125 μM. After an additional 20 hours incubation, the medium in each well was removed, and the wells were washed with 2 ml of PBS, followed by addition of 1 ml of PBS. Then, a fluorescent azide reactive molecule, DBCO-FAM (Broadpharm), was directly added to each well to produce a final concentration of 10 μM.

Plates with added DBCO-FAM were protected from bright light and incubated for 1 hour at room temperature. Supernatants in each well were removed, and each well was washed twice with 2 ml of PBS. Cells were then taken up with CellStripper (Thermo) reagent and transferred into 1.5 ml tubes. After pelleting and washing in PBS, cells were resuspended in 150 μl of PBS and counted. Defined numbers in 50 μl volumes in a 96-well Blackwell plate (Corning) were assayed for fluorescence in a fluorescent plate reader (Tecan).

Fluorescence arising from the reaction between DBCO-FAM and cell-surface azide label was also demonstrated by flow cytometry. In separate cultures, HeLa cells were added to wells of a 12-well plate at 10,000 cells/well, and cultured for 72 hours under normal conditions. The wells were washed with 1 ml of PBS, and 0.5 ml of DMEM-10% FBS was added. AzNAM was added to a final concentration of 125 μM, with cells receiving no azide-modified sugar as controls. After 20 hours, cells were harvested with CellStripper (250 μl/well), washed with PBS, and subjected to flow analysis.

By fluorescence readings in a 96-well plate format, it was demonstrated that cell-associated fluorescence increased as a function of the levels of AzNAM used in the initial labeling test. With flow analyses, marked and well-demarcated peak shifts were observed with the AzNAM-treated cells, which was corroborated by measuring fluorescence from equal numbers of cells in a fluorescent plate reader.

### Example 2: Anchoring an Active Molecule on the Surface of a Cell (Prophetic)

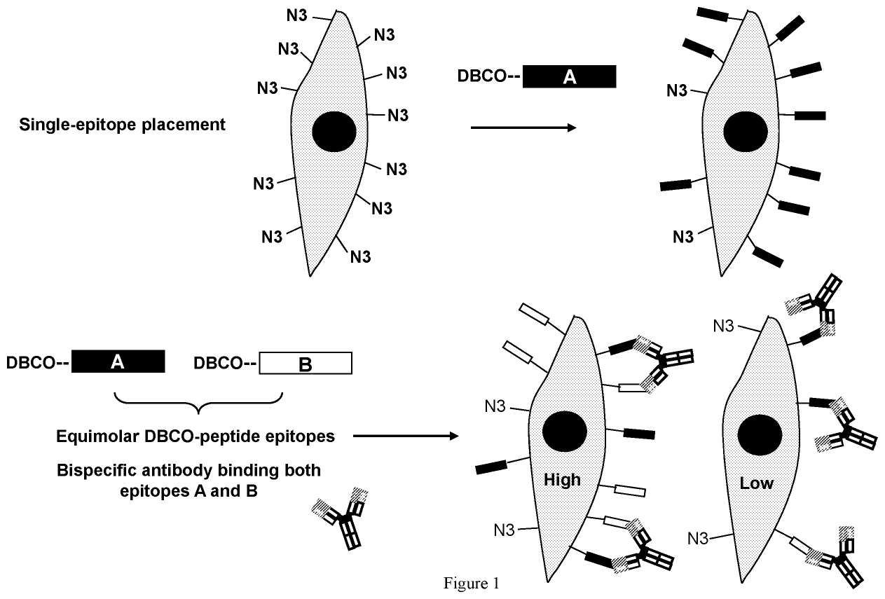

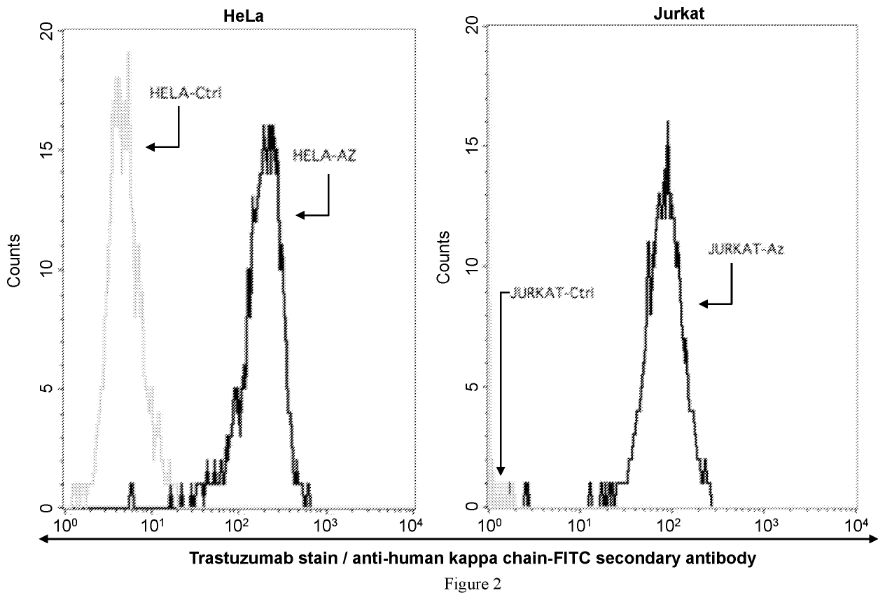

Metabolically labeled cells displaying azide groups are treated with DBCO-modified short peptide epitopes or mimotopes that are known targets for specific antibody recognition. Treatment of any cell capable of appropriately processing AzNAM compounds (with associated surface azide generation) will convert such a cell into a recognizable antibody target (see, FIG. 1, upper panel). High-density surface azide display can be exploited by coating target cells with equimolar mixes of two distinct DBCO-modified epitopes towards which a bispecific antibody is available (see, FIG. 1, lower panel). In such circumstances, cross-linking of the bispecific antibody (desired for high-affinity binding and some effector functions, such as antibody-dependent cellular cytotoxicity (ADCC)) is favored over comparable arrangements where the surface density is relatively low (see, FIG. 2).

### Example 3: Anchoring an Active Molecule on the Surface of a Cell Using Small Interactive Peptides (Prophetic)

Alternate methods for placement of proteins on cell surfaces by metabolic labeling include using small interactive peptides that are mutually interactive. When the NZ zipper is equipped with an N-terminal DBCO derivatization, the following sequence is produced: DBCO-SGGGSGASALKKELQANKKELAQLKWELQALKKELAQGAPGS (SEQ ID NO:85), where the DBCO can be conjugated by means of an N-terminal cysteine and maleimide chemistry. Treatment of cells metabolically displaying azides with the above DBCO-NZ sequence will result in coating with this peptide. In addition, any protein or peptide that is fused with the complementary CZ sequence EQLEKKLQALEKKLAQLEWK NQALEKKLAQ (SEQ ID NO:86) will specifically interact with the surface NZ.

### Example 4: Placement of a Trastuzumab Mimotope on the Surface of Azide-Modified Jurkat and HeLa Cells, and Demonstration by Flow Analysis

### Methods:

Jurkat and HeLa cells were cultured for three days in their optimal media (RPMI-10% FBS/DMEM-10% FBS, respectively) in 6-well plates to reach a state of sub-confluency. AzNAM (from 100 mM DMSO stock) was added to designated wells to a final volume of 100 μM. Control wells received equal volumes of DMSO only. After 24 hours, cells were harvested (directly for the non-adherent Jurkat cells, and with Cell Stripper reagent for HeLa cells), and washed in PBS. After counting and adjustment to concentrations of 10⁶ cells/ml each, each AzNAM/control preparation was treated with 10 μM of DBCO-FAM (control for demonstration of surface azide) or 10 μM of DBCO-Jmim, where Jmim indicates the trastuzumab mimotope used in this study: DBCO-SGGGSGGGQLGPYELWELSH (SEQ ID NO:87) After 1 hour of room temperature treatments with the DBCO reagents, cells were centrifuged and washed twice with PBS. Cells treated with the DBCO-Jmim peptide were then subjected to standard staining with Trastuzumab (BioVision), followed by secondary staining with FITC-conjugated goat-anti-human kappa chain antibody. During the staining procedure, the control washed DBCO-FAM treated cells were kept on ice. Finally, all cells were subjected to flow cytometric analysis.

### Results:

Flow analysis showed that both Jurkat and HeLa cells showed positive fluorescence as a consequence of metabolic labeling and DBCO-FAM treatment as expected. AzNAM-treated cells, but not DMSO controls, showed strong staining with trastuzumab for both Jurkat and HeLa cells (see, FIG. 2). This demonstrated the facility of epitope placement on cell surfaces, and conversion of the cells of interest into potential antibody targets.

### Example 5: Labeling Cells Having Surface Azide-Modified Sugars with Bifunctional Compounds (Prophetic)

Cells that have been metabolically labeled with surface azide-modified sugars (see, Example 1) can be subsequently reacted with the bifunctional compounds described herein. After such reactions have occurred and excess compound is removed, the portion of the bifunctional molecule that is a small molecule ligand that binds to an FKBP domain or FRB domain can be displayed on the surface of the cell and can be available for subsequent reactions with a CAR-T cell comprising an extracellular FKBP domain or FRB domain. Alternately, the portion of the bifunctional molecule that is a small molecule ligand that binds to an FKBP domain or FRB domain can first be allowed to react with the CART-T cell, after which the exposed azide reactive molecule of the bifunctional compound can be used for targeting the complex to the target cell surface labeled with azide-modified sugars.

In particular, any of the bifunctional compounds described herein can be used for the purposes of cell surface positioning of any of the CART-T cells described herein. Cells displaying azide moieties on surface glycan molecules (as in Example 1) can be treated with 1 mM of the bifunctional compound (initially solubilized in DMSO as a 100 mM stock solution and diluted accordingly to the final desired concentration) in serum-free RPMI medium for 2 hours at room temperature in the presence of 1 mg/ml bovine serum albumin (BSA) (Sigma) and 500 μg/ml salmon sperm DNA. This treatment is followed by centrifugation (5 minutes at 2000 rpm in an Eppendorf centrifuge), followed by two washes with serum-free RPMI medium, with resuspension in 100 μl of the same medium. Following this, a CART-T cell comprising an extracellular FKBP domain or FRB domain can be added to the bifunctional compound-modified target cells at a concentration of 1 pmol/μl, for a one hour incubation at room temperature. The cell preparations can be repelleted, washed twice with serum-free RPMI medium and once with PBS, with a final resuspension in 100 μl of PBS.

In an alternate embodiment, the CAR-T cell comprising an extracellular FKBP domain or FRB domain can be pre-incubated with excess bifunctional compound prior to exposure to the target cells displaying surface azide. The CAR-T cells in PBS (100 pmol) can be incubated with a 10-fold molar excess of the bifunctional compound for one hour at room temperature, followed by passage through a PBS-equilibrated P6 desalting column (Bio-Rad) to remove excess bifunctional compound. The resulting CAR-T cell-bifunctional compound can be used to treat target cells having surface azide, followed by washing steps as above.

### Example 6: Adapting CAR-T Systems for Recognition of Surface Azide (Prophetic)

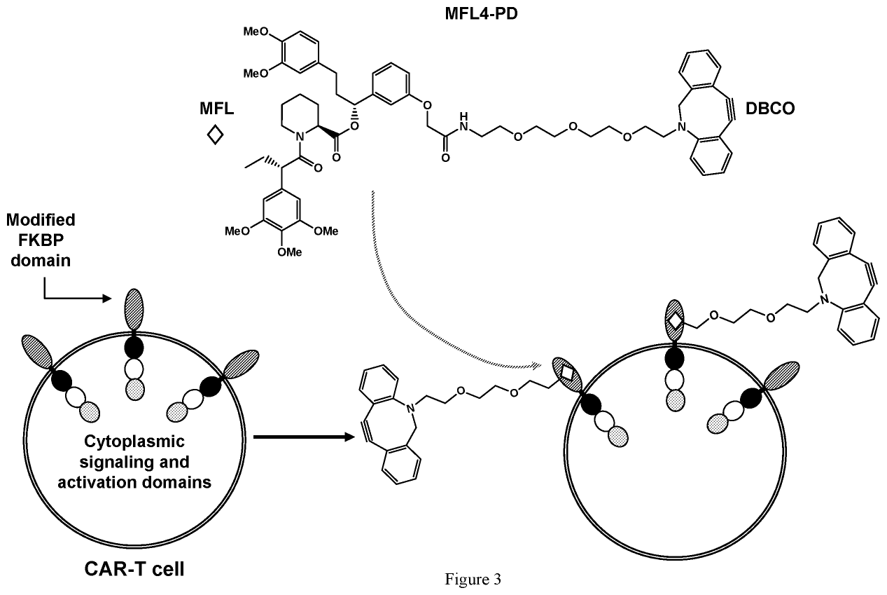

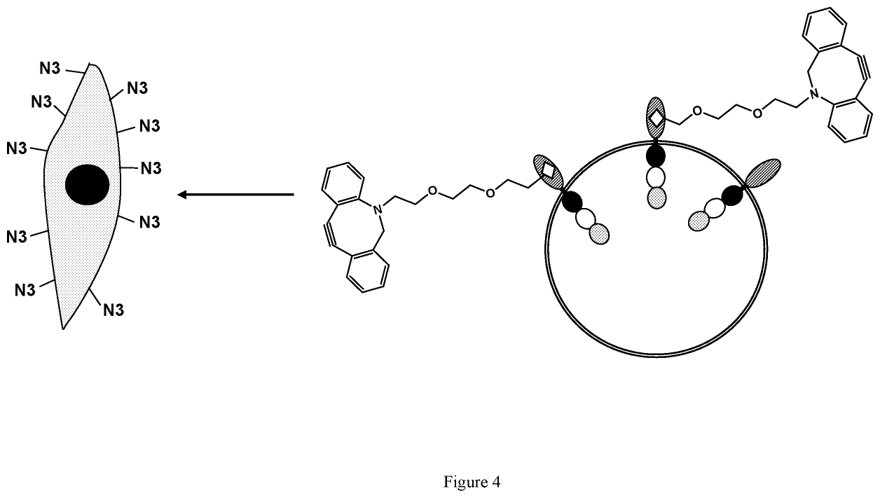

A CAR-T system is designed where T cells express a modified FKBP domain on the cell surface as a fusion with third-generation transmembrane/cytoplasmic signaling and activation domains (see, Wilkins et al., Human Gene Ther. Meth., 2017, 28, 61-66). Then, DBCO-MFL or BCN-MFL bifunctional compounds (or any of the bifunctional compounds described herein) can bind to such cells via the specific interaction of the MFL moiety with FKBP (see, FIG. 3). Subsequently, the DBCO or BCN moieties can react with surface azide-modified target cells (see, FIG. 4), thus directing the novel CAR-T to the desired target cell.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of anchoring an active molecule on the surface of a cell, the method comprising the steps:
a) contacting the cell with an azide-modified sugar; and
b) contacting the cell with an active molecule conjugated to an azide reactive molecule, wherein the azide reactive molecule is chemically reactable with the azide of the azide-modified sugar.

2. The method according to claim 1, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

3. The method according to claim 1, wherein the azide-modified sugar is AzNAM.

4. The method according to any one of claims 1 to 3, wherein the azide-modified sugar is acetylated at 1, 2, 3, or 4 positions.

5. The method according to any one of claims 1 to 4, wherein the azide reactive molecule is dibenzocyclooctyne (DBCO), bicyclo[6.1.0]nonyne (BCN), methyltetrazine, or trans-cyclooctene (TCO).

6. The method according to any one of claims 1 to 5, wherein the active molecule is a polypeptide or peptide, or mimotope thereof, that serves as an antigen for binding to an antibody, or antigen binding fragment thereof.

7. The method according to any one of claims 1 to 6, wherein the antigen binding fragment is single-chain antibody (ScFv), a Fab fragment, or a F(ab′)₂ fragment.

8. The method according to claim 6, wherein the polypeptide or peptide is HER-2 and the antibody is trastuzumab.

9. The method according to claim 8, wherein the HER-2 mimotope is a peptide comprising the amino acid sequence QLGPYELWELSH (SEQ ID NO:1) or LLGPYELWELSH (SEQ ID NO:2).

10. The method according to claim 8, wherein the HER-2 mimotope is a polypeptide comprising the formula: SerGlyGlyGlySerGlyGlyGlyGlnLeuXaa¹ProTyrGluXaa²TrpGluLeu Xaa³His (SEQ ID NO:3), wherein one of: a) Xaa¹ is Cys, Xaa² is Leu, and Xaa³ is Ser (SEQ ID NO:4); b) Xaa¹ is Gly, Xaa² is Cys, and Xaa³ is Ser (SEQ ID NO:5); c) Xaa¹ is Gly, Xaa² is Leu, and Xaa³ is Cys (SEQ ID NO:6), or d) Xaa¹ is Gly, Xaa² is Leu, and Xaa³ is Ser (SEQ ID NO:87).

11. The method according to claim 8, wherein the HER-2 mimotope is a polypeptide comprising the formula: SerGlyGlyGlySerGlyGlyGlyGlnXaa¹LeuXaa²GlyXaa³ProXaa⁴Tyr Xaa⁵GluXaa⁶LeuXaa⁷TrpXaa⁸GluXaa⁹LeuXaa¹⁰SerXaa¹¹His (SEQ ID NO:7), wherein one of: a) Xaa¹ is Cys and Xaa², Xaa³, Xaa⁴, Xaa⁵, Xaa⁶, Xaa⁷, Xaa⁸, Xaa⁹, Xaa¹⁰, and Xaa¹¹ are absent (SEQ ID NO:8); b) Xaa² is Cys and Xaa¹, Xaa³, Xaa⁴, Xaa⁵, Xaa⁶, Xaa⁷, Xaa⁸, Xaa⁹, Xaa¹⁰, and Xaa¹¹ are absent (SEQ ID NO:9); c) Xaa³ is Cys and Xaa¹, Xaa², Xaa⁴, Xaa⁵, Xaa⁶, Xaa⁷, Xaa⁸, Xaa⁹, Xaa¹⁰, and Xaa¹¹ are absent (SEQ ID NO:10); d) Xaa⁴ is Cys and Xaa¹, Xaa², Xaa³, Xaa⁵, Xaa⁶, Xaa⁷, Xaa⁸, Xaa⁹, Xaa¹⁰, and Xaa¹¹ are absent (SEQ ID NO:11); e) Xaa⁵ is Cys and Xaa¹, Xaa², Xaa³, Xaa⁴, Xaa⁶, Xaa⁷, Xaa⁸, Xaa⁹, Xaa¹⁰, and Xaa¹¹ are absent (SEQ ID NO:12); f) Xaa⁶ is Cys and Xaa¹, Xaa², Xaa³, Xaa⁴, Xaa⁵, Xaa⁷, Xaa⁸, Xaa⁹, Xaa¹⁰, and Xaa¹¹ are absent (SEQ ID NO:13); g) Xaa⁷ is Cys and Xaa¹, Xaa², Xaa³, Xaa⁴, Xaa⁵, Xaa⁶, Xaa⁸, Xaa⁹, Xaa¹⁰, and Xaa¹¹ are absent (SEQ ID NO:14); h) Xaa⁸ is Cys and Xaa¹, Xaa², Xaa³, Xaa⁴, Xaa⁵, Xaa⁶, Xaa⁷, Xaa⁹, Xaa¹⁰, and Xaa¹¹ are absent (SEQ ID NO:15); i) Xaa⁹ is Cys and Xaa¹, Xaa², Xaa³, Xaa⁴, Xaa⁵, Xaa⁶, Xaa⁷, Xaa⁸, Xaa¹⁰, and Xaa¹¹are absent (SEQ ID NO:16); j) Xaa¹⁰ is Cys and Xaa¹, Xaa², Xaa³, Xaa⁴, Xaa⁵, Xaa⁶, Xaa⁷, Xaa⁸, Xaa⁹, and Xaa¹¹ are absent (SEQ ID NO:17); or k) Xaa¹¹ is Cys and Xaa¹, Xaa², Xaa³, Xaa⁴, Xaa⁵, Xaa⁶, Xaa⁷, Xaa⁸, Xaa⁹, and Xaa¹⁰ are absent (SEQ ID NO:18).

12. The method according to claim 6, wherein the polypeptide or peptide is the F glycoprotein of Respiratory Syncytial Virus (RSV) and the antibody is palivizumab or motavizumab.

13. The method according to claim 12, wherein the F glycoprotein of RSV mimotope is a peptide comprising the amino acid sequence NSELLSLINDMPITNDQKKLMSNN (SEQ ID NO:19).

14. The method according to claim 6, wherein the polypeptide or peptide is Epidermal Growth Factor Receptor (EGFR) and the antibody is panitumumab.

15. The method according to claim 14, wherein the EGFR mimotope is a peptide comprising the amino acid sequence IYPPLLRTSQAM (SEQ ID NO:20), AYPPYLRSMTLY (SEQ ID NO:21), YPPAERTYSTNY (SEQ ID NO:22), CPKWDAARC (SEQ ID NO:23), or CGPTRWRSC (SEQ ID NO:24).

16. The method according to claim 6, wherein the polypeptide or peptide is the Vi antigen of Salmonella enterica and the antibody is ATVi.

17. The method according to claim 16, wherein the Vi antigen mimotope is a peptide comprising the amino acid sequence TSHHDSHGLHRV (SEQ ID NO:25), TSHHDSHGDHHV (SEQ ID NO:26), TSHHDSHGVHRV (SEQ ID NO:27), TSHHDSHDLHRV (SEQ ID NO:28), TSHHDYHGLHRV (SEQ ID NO:29), ENHSPVNIAHKL (SEQ ID NO:30), ENHSPVNIAHKV (SEQ ID NO:31), ENHSPVNIDHKL (SEQ ID NO:32), EDHSPVNIDHKL (SEQ ID NO:33), ENHYPLHAAHRI (SEQ ID NO:34), ESHQHVHDLVFL (SEQ ID NO:35), PGHHDFVGLHHL (SEQ ID NO:36), ENHYPVNIAHKL (SEQ ID NO:37), or DNHSPVNIAHKL (SEQ ID NO:38).

18. The method according to claim 6, wherein the polypeptide or peptide is a polypeptide on H5N1 Influenza Virus and the antibody is AVFluIgG01.

19. The method according to claim 18, wherein the H5N1 Influenza Virus mimotope is a peptide comprising the amino acid sequence YINPHMYWMSVA (SEQ ID NO:39), HTPPPQPYRTHI (SEQ ID NO:40), TFWVQTAKPNPL (SEQ ID NO:41), GHPSKTSGHPLT (SEQ ID NO:42), TYVNIVLYDDVE (SEQ ID NO:43), TTNFLNHAIAHK (SEQ ID NO:44), YYNPSPPNPRTQ (SEQ ID NO:45), TESPQYIALSFH (SEQ ID NO:46), HWYDWLTRYSHL (SEQ ID NO:47), ATYTTDAQSYHM (SEQ ID NO:48), DHYWHRSNTLSH (SEQ ID NO:49), VTSHDLKKSGTW (SEQ ID NO:50), WEFAYKNTRYYW (SEQ ID NO:51), SWTSLPLHEAIH (SEQ ID NO:52), TLAHTHTSTSSF (SEQ ID NO:53), WHWSFFASPLPA (SEQ ID NO:54), WHWNARNWSSQQ (SEQ ID NO:55), CWTSLPLHEAIH (SEQ ID NO:56), VPTECSGRTSCT (SEQ ID NO:57), WSNHWWHSKWAI (SEQ ID NO:58), HIWNWSNWTQWT (SEQ ID NO:59), HIFHNTHWWQRW (SEQ ID NO:60), TNYDYIPDTQNT (SEQ ID NO:61), SWSSHSNSTPTSYNTNQTQNPTSTSTNQPNNN (SEQ ID NO:62), or NHEKIPKSSWSSHWKYNTNQEDNKTIKPNDNEYKVK (SEQ ID NO:63).

20. The method according to claim 6, wherein the polypeptide or peptide is CD147 and the antibody is metuximab.

21. The method according to claim 20, wherein the CD147 mimotope is a peptide comprising the amino acid sequence YPHFHKHTLRGH (SEQ ID NO:64), YPHFHKHSLRGQ (SEQ ID NO:65), DHKPFKPTHRTL (SEQ ID NO:66), FHKPFKPTHRTL (SEQ ID NO:67), QSSCHKHSVRGR (SEQ ID NO:68), QSSFSNHSVRRR (SEQ ID NO:69), or DFDVSFLSARMR (SEQ ID NO:70).

22. The method according to claim 6, wherein the polypeptide or peptide is a protein on Schistosoma mansoni and the antibody is 152-66-9b.

23. The method according to claim 22, wherein the protein on Schistosoma mansoni mimotope is a peptide comprising the amino acid sequence VLLRRIGG (SEQ ID NO:71), HLLRLSEI (SEQ ID NO:72), SLLTYMKM (SEQ ID NO:73), or YLLQKLRN (SEQ ID NO:74).

24. A method of anchoring at least two active molecules on the surface of a cell, the method comprising the steps:
a) contacting the cell with an azide-modified sugar;
b) contacting the cell with a first active molecule conjugated to a first azide reactive molecule, wherein the first azide reactive molecule is chemically reactable with the azide of the azide-modified sugar; and
c) contacting the cell with a second active molecule conjugated to a second azide reactive molecule, wherein the second azide reactive molecule is chemically reactable with the azide of the azide-modified sugar.

25. The method according to claim 24, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

26. The method according to claim 24, wherein the azide-modified sugar is AzNAM.

27. The method according to any one of claims 24 to 26, wherein the azide-modified sugar is acetylated at 1, 2, 3, or 4 positions.

28. The method according to any one of claims 24 to 27, wherein the first azide reactive molecule and second azide reactive molecule are, independently, dibenzocyclooctyne (DBCO), bicyclo[6.1.0]nonyne (BCN), methyltetrazine, or trans-cyclooctene (TCO).

29. The method according to any one of claims 24 to 28, wherein the first active molecule and second active molecule are, independently, a polypeptide or peptide, or mimotope thereof, that serve as an antigen for binding to a bi-specific antibody, or antigen binding fragment thereof.

30. The method according to any one of claims 24 to 29, wherein the antigen binding fragment is single-chain antibody (ScFv), a Fab fragment, or a F(ab′)₂ fragment.

31. The method according to any one of claims 24 to 29, wherein the first active molecule and second active molecule are, independently, a mimotope for HER-2, the F glycoprotein of RSV, EGFR, the Vi antigen of Salmonella enterica, a polypeptide on H5N1 Influenza Virus, CD147, or a protein on Schistosoma mansoni.

32. A method of anchoring an active molecule on the surface of a cell, the method comprising the steps:
a) contacting the cell with an azide-modified sugar;
b) contacting the cell with a first small interactive peptide conjugated to an azide reactive molecule, wherein the azide reactive molecule is chemically reactable with the azide of the azide-modified sugar; and
c) contacting the cell with a second small interactive peptide conjugated to an active molecule, wherein the first small interactive peptide interacts with the second small interactive peptide.

33. The method according to claim 32, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

34. The method according to claim 32, wherein the azide-modified sugar is AzNAM.

35. The method according to any one of claims 32 to 34, wherein the azide-modified sugar is acetylated at 1, 2, 3, or 4 positions.

36. The method according to any one of claims 32 to 35, wherein the azide reactive molecule is dibenzocyclooctyne (DBCO), bicyclo[6.1.0]nonyne (BCN), methyltetrazine, or trans-cyclooctene (TCO).

37. The method according to any one of claims 32 to 36, wherein the active molecule is a polypeptide or peptide, or mimotope thereof, that serves as an antigen for binding to an antibody, or antigen binding fragment thereof.

38. The method according to any one of claims 32 to 37, wherein the antigen binding fragment is single-chain antibody (ScFv), a Fab fragment, or a F(ab′)₂ fragment.

39. The method according to any one of claims 32 to 38, wherein the first small interactive peptide and the second small interactive peptide are chosen from jun/fos, mad/max, myc/max, and NZ/CZ zipper domains.

40. The method according to claim 39, wherein one of the first small interactive peptide and the second small interactive peptide is a c-jun polypeptide comprising an amino acid sequence chosen from CSGGASLERIARLEEKVKTLKAQNSELASTANMLREQVAQLK QKGAP (SEQ ID NO:75), SGASLERIARLEEKVKTLKAQNSELASTANMLREQVA QLKQKGAPSGGC (SEQ ID NO:76), CSGGASLERIARLEEKVKSFKAQNSENASTA NMLREQVAQLKQKGAP (SEQ ID NO:77), SGASLERIARLEEKVKSFKAQNSENAS TANMLREQVAQLKQKGAPSGGC (SEQ ID NO:78), CSGASLERIARLEEKVKSFKA QNSENASTANMLREQVAQLKQKGAP (SEQ ID NO:79), and GASLERIARLEEKV KTLKAQNSELASTANMLREQVAQLKQKGAPSGGC (SEQ ID NO:80), and the other of the first small interactive peptide and the second small interactive peptide is a c-fos polypeptide comprising an amino acid sequence chosen from ASRELTDTLQAETDQLEDE KSALQTEIANLLKEKEKLEGAP (SEQ ID NO:81), ASRETDTLQAETDQLEDEKSA LQTEIANLLKEKEKLEGAP (SEQ ID NO:82), and SGASRELTDTLQAETDQLEDE KSALQTEIANLLKEKEKLEGAP (SEQ ID NO:83).

41. The method according to claim 39, wherein one of the first small interactive peptide and the second small interactive peptide is an NZ domain comprising an amino acid sequence chosen from ALKKELQANKKELAQLKWELQALKKELAQ (SEQ ID NO:84) and SGGGSGASALKKELQANKKELAQLKWELQALKKELAQGAPGS (SEQ ID NO:85), and the other of the first small interactive peptide and the second small interactive peptide is a CZ domain comprising an amino acid sequence is EQLEKKLQALEKKLAQLEWKNQALEKKLAQ (SEQ ID NO:86).

42. The method according to any one of claims 32 to 41, wherein the active molecule is a mimotope for HER-2, the F glycoprotein of RSV, EGFR, the Vi antigen of Salmonella enterica, a polypeptide on H5N1 Influenza Virus, CD147, or a protein on Schistosoma mansoni.

43. A method of enhancing an immune response to a target cell in a human, the method comprising the steps:
a) contacting the target cell in the human with an azide-modified sugar;
b) introducing into the human a CAR-T cell, wherein the CAR-T cell comprises an extracellular FK506-binding protein (FKBP) domain or FRB domain functionally linked to a cytoplasmic signaling domain of the CART-T cell; and
c) contacting the target cell in the human with a bifunctional FKBP or FRB domain binding compound, wherein a first portion of the bifunctional FKBP domain binding compound interacts with the FKBP domain on the CAR-T cell, and a second portion of the bifunctional FKBP or FRB domain binding compound interacts with the azide of the azide-modified sugar on the target cell.

44. The method according to claim 43, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

45. The method according to claim 43, wherein the azide-modified sugar is AzNAM.

46. The method according to any one of claims 43 to 45, wherein the azide-modified sugar is acetylated at 1, 2, 3, or 4 positions.

47. The method according to any one of claims 43 to 46, wherein the second portion of the bifunctional FKBP or FRB domain binding compound comprises an azide reactive molecule.

48. The method according to any one of claims 43 to 47, wherein the CAR-T cell comprises an extracellular FKBP domain functionally linked to the cytoplasmic signaling domain of the CART-T cell.

49. The method according to claim 48, wherein the FKBP domain is a mutant FKBP domain.

50. The method according to claim 49, wherein the mutant FKBP domain is the F36V FKBP mutant domain comprising the amino acid sequence GVQVETISPGDGRTFPKRGQT CVVHYTGMLEDGKKVDSSRDRNKPFKFMLGKQEVIRGWEEGVAQMSVGQRAKLTI SPDYAYGATGHPGIIPPHATLVFDVELLKLE (SEQ ID NO:88) or MGVQVETISPGDG RTFPKRGQTCVVHYTGMLEDGKKVDSSRDRNKPFKFMLGKQEVIRGWEEGVAQMS VGQRAKLTISPDYAYGATGHPGIIPPHATLVFDVELLKLE (SEQ ID NO:89).

51. The method according to claim 49, wherein the mutant FKBP domain comprises a C22S, C22A, or C22V substitution.

52. The method according to any one of claims 43 to 47, wherein the CAR-T cell comprises an extracellular FRB domain functionally linked to the cytoplasmic signaling domain of the CART-T cell.

53. The method according to claim 52, wherein the FRB domain comprises a C61S, C61A, or C61V substitution.

54. The method according to any one of claims 43 to 53, wherein the bifunctional FKBP or FRB domain binding compound comprises the formula: 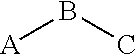
wherein:
A is a small molecule ligand that binds to an FKBP domain or FRB domain;
B is a chemical linker chosen from an alkyl, an alkenyl, an amide, an ester, a thioester, a ketone, an ether, a thioether, a disulfide, an ethylene glycol unit, a cycloalkyl, a benzyl, a heterocyclic, a maleimidyl, a hydrazone, a urethane, an azole, an imine, a haloalkyl, or a carbamate, or any combination thereof; and
C is an azide reactive molecule chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, or a quadricyclane.

55. The method according to claim 54, wherein the small molecule ligand comprises

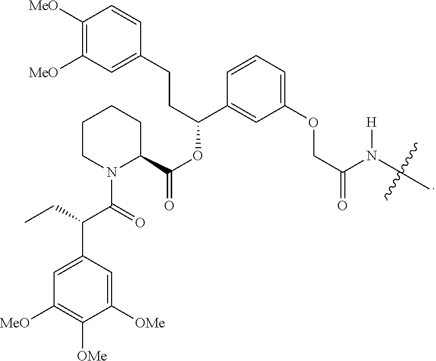 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

56. The method according to claim 54 or claim 55, wherein the chemical linker is an alkyl group or an ethylene glycol unit.

57. The method according to claim 56, wherein the chemical linker is an alkyl group.

58. The method according to claim 57, wherein the chemical linker is a C₂-C₁₆alkyl group.

59. The method according to claim 57, wherein the chemical linker is a C₄-C₁₂alkyl group or a C₄-C₁₆alkyl group.

60. The method according to claim 57, wherein the chemical linker is a C₄-C₁alkyl group.

61. The method according to claim 57, wherein the chemical linker is C₄alkyl group or C₁₀alkyl group.

62. The method according to claim 56, wherein the chemical linker is an ethylene glycol unit.

63. The method according to claim 62, wherein the chemical linker is a polyethylene glycol (PEG) unit.

64. The method according to claim 63, wherein the PEG is PEG2 to PEG16.

65. The method according to claim 63, wherein the PEG is PEG2, PEG3, or PEG4.

66. The method according to any one of claims 54 to 65, wherein the azide reactive molecule is chosen from a cyclooctyne, a cyclooctene, and a tetrazine.

67. The method according to claim 66, wherein the cyclooctyne is dibenzocyclooctyne (DECO), bicyclo[6.1.0]nonyne (BCN), monofluorinated cyclooctyne, ditluorocyclooctyne, dimethoxyazacyclooctyne, dibenzoazacyclooctyne, biarylazacyclooctynone, 2,3,6,7-tetramethoxy-1-dibenzocyclooctyne, sulfonylated dibenzocyclooctyne, carboxymethylmonobenzocyclooctyne, or pyrrolocyclooctyne.

68. The method according to claim 66, wherein the cyclooctene is trans-cyclooctene (TCO).

69. The method according to claim 66, wherein the tetrazine is methyltetrazine, diphenyltetrazine, 3,6-di-(2-pyridyl)-s-tetrazine, 3,6-diphenyl-s-tetrazine, 3-(5-aminopyridin-2-yl)-6-(pyridin-2-yl)-s-tetrazine, or N-benzoyl-3-(5-aminopyridin-2-yl)-6-(pyridin-2-yl)-s-tetrazine.

70. The method according to claim 55, wherein the chemical linker is an alkyl group or an ethylene glycol unit, and the azide reactive molecule is chosen from a cyclooctyne, a cyclooctene, and a tetrazine.

71. The method according to claim 55, wherein the chemical linker is a C₂-C₁₆alkyl group, or a polyethylene glycol unit which is PEG2 to PEG16, and the azide reactive molecule is DBCO, BCN, TCO, or methyltetrazine.

72. The method according to claim 55, wherein the chemical linker is a C₄-C₁₀alkyl group or a polyethylene glycol unit which is PEG2, PEG3, or PEG4, and the azide reactive molecule is DBCO, BCN, TCO, or methyltetrazine.

73. The method according to claim 55, wherein the chemical linker is C₄alkyl group, C₁₀alkyl group, or PEG3, and the azide reactive molecule is DBCO or 3CN.

74. The method according to claim 55, wherein the bifunctional FKBP or FRB domain binding compound comprises the formula:

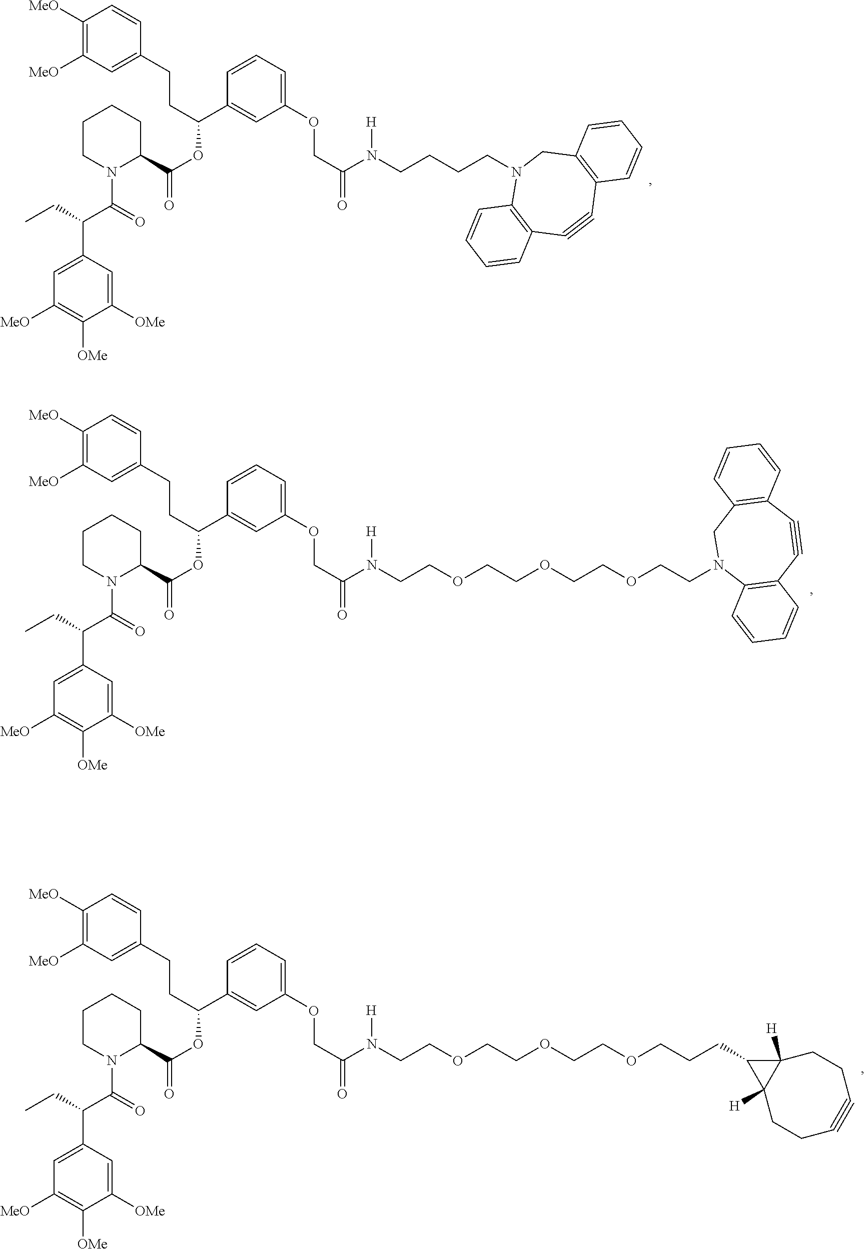 [SMILES: CC[C@H](C(=O)N1CCCC[C@H]1C(=O)O[C@H](CCc1ccc(OC)c(OC)c1)c1cccc(OCC(=O)NCCCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCC[C@@H]2[C@@H]3CCC#CCC[C@@H]32)c1)c1cc(OC)c(OC)c(OC)c1]

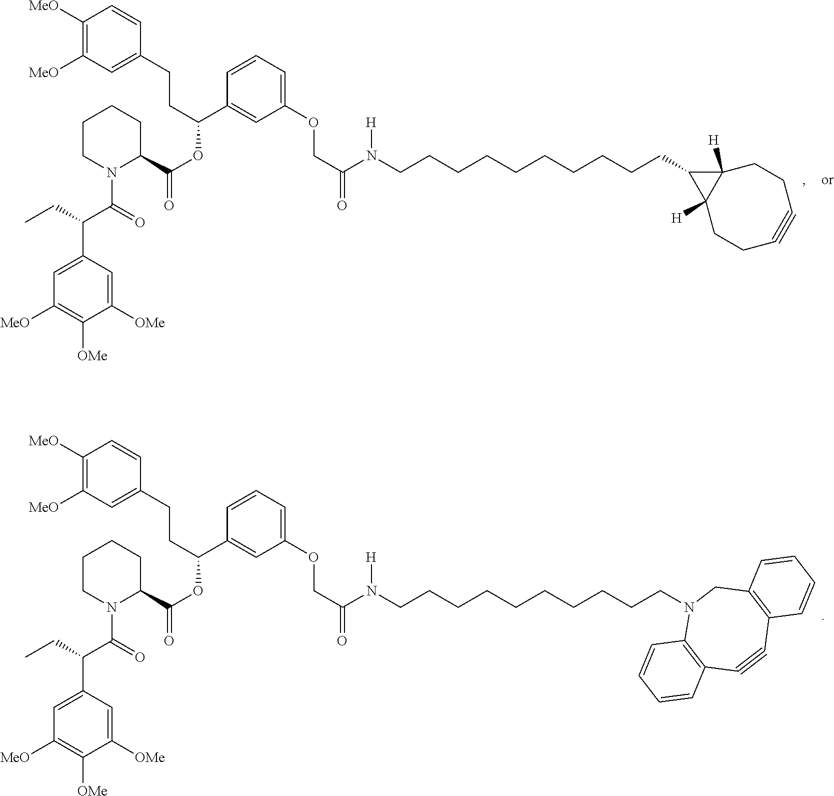 [SMILES: CC[C@H](C(=O)N1CCCC[C@H]1C(=O)O[C@H](CCc1ccc(OC)c(OC)c1)c1cccc(OCC(=O)NCCCCCCCCCC[C@@H]2[C@@H]3CCC#CCC[C@@H]32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCCCCCCCCCN2Cc3ccccc3C#Cc3ccccc32)c1)c1cc(OC)c(OC)c(OC)c1]

75. The method according to any one of claims 43 to 74, wherein the CAR-T cell comprising the extracellular FKBP domain or FRB domain functionally linked to the cytoplasmic signaling domain of the CART-T cell is pre-incubated with excess bifunctional compound prior to their contacting the cells.


SEQUENCE LISTING (100 sequences)

SEQ ID NO: 1 - protein, 12 aa, Artificial Sequence
  Description of Artificial Sequence Synthetic peptide
QLGPYELWELSH

SEQ ID NO: 2 - protein, 12 aa, Artificial Sequence
  Description of Artificial Sequence Synthetic peptide
LLGPYELWELSH

SEQ ID NO: 3 - protein, 20 aa, Artificial Sequence
  Description of Artificial Sequence Synthetic peptide
  MOD_RES (11)..(11): Any amino acid
  MOD_RES (15)..(15): Any amino acid
  MOD_RES (19)..(19): Any amino acid
SGGGSGGGQLXPYEXWELXH

SEQ ID NO: 4 - protein, 20 aa, Artificial Sequence
  Description of Artificial Sequence Synthetic peptide
SGGGSGGGQLCPYELWELSH

SEQ ID NO: 5 - protein, 20 aa, Artificial Sequence
  Description of Artificial Sequence Synthetic peptide
SGGGSGGGQLGPYECWELSH

SEQ ID NO: 6 - protein, 20 aa, Artificial Sequence
  Description of Artificial Sequence Synthetic peptide
SGGGSGGGQLGPYELWELCH

SEQ ID NO: 7 - protein, 31 aa, Artificial Sequence
  Description of Artificial Sequence Synthetic polypeptide
  MOD_RES (10)..(10): Any amino acid
  MOD_RES (12)..(12): Any amino acid
  MOD_RES (14)..(14): Any amino acid
  MOD_RES (16)..(16): Any amino acid
  MOD_RES (18)..(18): Any amino acid
  MOD_RES (20)..(20): Any amino acid
  MOD_RES (22)..(22): Any amino acid
  MOD_RES (24)..(24): Any amino acid
  MOD_RES (26)..(26): Any amino acid
  MOD_RES (28)..(28): Any amino acid
  MOD_RES (30)..(30): Any amino acid
SGGGSGGGQXLXGXPXYXEXLXWXEXLXSXH

SEQ ID NO: 8 - protein, 21 aa, Artificial Sequence
  Description of Artificial Sequence Synthetic peptide
SGGGSGGGQCLGPYELWELSH

SEQ ID NO: 9 - protein, 21 aa, Artificial Sequence
  Description of Artificial Sequence Synthetic peptide
SGGGSGGGQLCGPYELWELSH

SEQ ID NO: 10 - protein, 21 aa, Artificial Sequence
  Description of Artificial Sequence Synthetic peptide
SGGGSGGGQLGCPYELWELSH

SEQ ID NO: 11 - protein, 21 aa, Artificial Sequence
  Description of Artificial Sequence Synthetic peptide
SGGGSGGGQLGPCYELWELSH

SEQ ID NO: 12 - protein, 21 aa, Artificial Sequence
  Description of Artificial Sequence Synthetic peptide
SGGGSGGGQLGPYCELWELSH

SEQ ID NO: 13 - protein, 21 aa, Artificial Sequence
  Description of Artificial Sequence Synthetic peptide
SGGGSGGGQLGPYECLWELSH

SEQ ID NO: 14 - protein, 21 aa, Artificial Sequence
  Description of Artificial Sequence Synthetic peptide
SGGGSGGGQLGPYELCWELSH

SEQ ID NO: 15 - protein, 21 aa, Artificial Sequence
  Description of Artificial Sequence Synthetic peptide
SGGGSGGGQLGPYELWCELSH

SEQ ID NO: 16 - protein, 21 aa, Artificial Sequence
  Description of Artificial Sequence Synthetic peptide
SGGGSGGGQLGPYELWECLSH

SEQ ID NO: 17 - protein, 21 aa, Artificial Sequence
  Description of Artificial Sequence Synthetic peptide
SGGGSGGGQLGPYELWELCSH

SEQ ID NO: 18 - protein, 21 aa, Artificial Sequence
  Description of Artificial Sequence Synthetic peptide
SGGGSGGGQLGPYELWELSCH

SEQ ID NO: 19 - protein, 24 aa, Artificial Sequence
  Description of Artificial Sequence Synthetic peptide
NSELLSLINDMPITNDQKKLMSNN

SEQ ID NO: 20 - protein, 12 aa, Artificial Sequence
  Description of Artificial Sequence Synthetic peptide
IYPPLLRTSQAM

SEQ ID NO: 21 - protein, 12 aa, Artificial Sequence
  Description of Artificial Sequence Synthetic peptide
AYPPYLRSMTLY

SEQ ID NO: 22 - protein, 12 aa, Artificial Sequence
  Description of Artificial Sequence Synthetic peptide
YPPAERTYSTNY

SEQ ID NO: 23 - protein, 9 aa, Artificial Sequence
  Description of Artificial Sequence Synthetic peptide
CPKWDAARC

SEQ ID NO: 24 - protein, 9 aa, Artificial Sequence
  Description of Artificial Sequence Synthetic peptide
CGPTRWRSC

SEQ ID NO: 25 - protein, 12 aa, Artificial Sequence
  Description of Artificial Sequence Synthetic peptide
TSHHDSHGLHRV

SEQ ID NO: 26 - protein, 12 aa, Artificial Sequence
  Description of Artificial Sequence Synthetic peptide
TSHHDSHGDHHV

SEQ ID NO: 27 - protein, 12 aa, Artificial Sequence
  Description of Artificial Sequence Synthetic peptide
TSHHDSHGVHRV

SEQ ID NO: 28 - protein, 12 aa, Artificial Sequence
  Description of Artificial Sequence Synthetic peptide
TSHHDSHDLHRV

SEQ ID NO: 29 - protein, 12 aa, Artificial Sequence
  Description of Artificial Sequence Synthetic peptide
TSHHDYHGLHRV

SEQ ID NO: 30 - protein, 12 aa, Artificial Sequence
  Description of Artificial Sequence Synthetic peptide
ENHSPVNIAHKL

SEQ ID NO: 31 - protein, 12 aa, Artificial Sequence
  Description of Artificial Sequence Synthetic peptide
ENHSPVNIAHKV

SEQ ID NO: 32 - protein, 12 aa, Artificial Sequence
  Description of Artificial Sequence Synthetic peptide
ENHSPVNIDHKL

SEQ ID NO: 33 - protein, 12 aa, Artificial Sequence
  Description of Artificial Sequence Synthetic peptide
EDHSPVNIDHKL

SEQ ID NO: 34 - protein, 12 aa, Artificial Sequence
  Description of Artificial Sequence Synthetic peptide
ENHYPLHAAHRI

SEQ ID NO: 35 - protein, 12 aa, Artificial Sequence
  Description of Artificial Sequence Synthetic peptide
ESHQHVHDLVFL

SEQ ID NO: 36 - protein, 12 aa, Artificial Sequence
  Description of Artificial Sequence Synthetic peptide
PGHHDFVGLHHL

SEQ ID NO: 37 - protein, 12 aa, Artificial Sequence
  Description of Artificial Sequence Synthetic peptide
ENHYPVNIAHKL

SEQ ID NO: 38 - protein, 12 aa, Artificial Sequence
  Description of Artificial Sequence Synthetic peptide
DNHSPVNIAHKL

SEQ ID NO: 39 - protein, 12 aa, Artificial Sequence
  Description of Artificial Sequence Synthetic peptide
YINPHMYWMSVA

SEQ ID NO: 40 - protein, 12 aa, Artificial Sequence
  Description of Artificial Sequence Synthetic peptide
HTPPPQPYRTHI

SEQ ID NO: 41 - protein, 12 aa, Artificial Sequence
  Description of Artificial Sequence Synthetic peptide
TFWVQTAKPNPL

SEQ ID NO: 42 - protein, 12 aa, Artificial Sequence
  Description of Artificial Sequence Synthetic peptide
GHPSKTSGHPLT

SEQ ID NO: 43 - protein, 12 aa, Artificial Sequence
  Description of Artificial Sequence Synthetic peptide
TYVNIVLYDDVE

SEQ ID NO: 44 - protein, 12 aa, Artificial Sequence
  Description of Artificial Sequence Synthetic peptide
TTNFLNHAIAHK

SEQ ID NO: 45 - protein, 12 aa, Artificial Sequence
  Description of Artificial Sequence Synthetic peptide
YYNPSPPNPRTQ

SEQ ID NO: 46 - protein, 12 aa, Artificial Sequence
  Description of Artificial Sequence Synthetic peptide
TESPQYIALSFH

SEQ ID NO: 47 - protein, 12 aa, Artificial Sequence
  Description of Artificial Sequence Synthetic peptide
HWYDWLTRYSHL

SEQ ID NO: 48 - protein, 12 aa, Artificial Sequence
  Description of Artificial Sequence Synthetic peptide
ATYTTDAQSYHM

SEQ ID NO: 49 - protein, 12 aa, Artificial Sequence
  Description of Artificial Sequence Synthetic peptide
DHYWHRSNTLSH

SEQ ID NO: 50 - protein, 12 aa, Artificial Sequence
  Description of Artificial Sequence Synthetic peptide
VTSHDLKKSGTW

SEQ ID NO: 51 - protein, 12 aa, Artificial Sequence
  Description of Artificial Sequence Synthetic peptide
WEFAYKNTRYYW

SEQ ID NO: 52 - protein, 12 aa, Artificial Sequence
  Description of Artificial Sequence Synthetic peptide
SWTSLPLHEAIH

SEQ ID NO: 53 - protein, 12 aa, Artificial Sequence
  Description of Artificial Sequence Synthetic peptide
TLAHTHTSTSSF

SEQ ID NO: 54 - protein, 12 aa, Artificial Sequence
  Description of Artificial Sequence Synthetic peptide
WHWSFFASPLPA

SEQ ID NO: 55 - protein, 12 aa, Artificial Sequence
  Description of Artificial Sequence Synthetic peptide
WHWNARNWSSQQ

SEQ ID NO: 56 - protein, 12 aa, Artificial Sequence
  Description of Artificial Sequence Synthetic peptide
CWTSLPLHEAIH

SEQ ID NO: 57 - protein, 12 aa, Artificial Sequence
  Description of Artificial Sequence Synthetic peptide
VPTECSGRTSCT

SEQ ID NO: 58 - protein, 12 aa, Artificial Sequence
  Description of Artificial Sequence Synthetic peptide
WSNHWWHSKWAI

SEQ ID NO: 59 - protein, 12 aa, Artificial Sequence
  Description of Artificial Sequence Synthetic peptide
HIWNWSNWTQWT

SEQ ID NO: 60 - protein, 12 aa, Artificial Sequence
  Description of Artificial Sequence Synthetic peptide
HIFHNTHWWQRW

SEQ ID NO: 61 - protein, 12 aa, Artificial Sequence
  Description of Artificial Sequence Synthetic peptide
TNYDYIPDTQNT

SEQ ID NO: 62 - protein, 32 aa, Artificial Sequence
  Description of Artificial Sequence Synthetic polypeptide
SWSSHSNSTPTSYNTNQTQNPTSTSTNQPNNN

SEQ ID NO: 63 - protein, 36 aa, Artificial Sequence
  Description of Artificial Sequence Synthetic polypeptide
NHEKIPKSSWSSHWKYNTNQEDNKTIKPNDNEYKVK

SEQ ID NO: 64 - protein, 12 aa, Artificial Sequence
  Description of Artificial Sequence Synthetic peptide
YPHFHKHTLRGH

SEQ ID NO: 65 - protein, 12 aa, Artificial Sequence
  Description of Artificial Sequence Synthetic peptide
YPHFHKHSLRGQ

SEQ ID NO: 66 - protein, 12 aa, Artificial Sequence
  Description of Artificial Sequence Synthetic peptide
DHKPFKPTHRTL

SEQ ID NO: 67 - protein, 12 aa, Artificial Sequence
  Description of Artificial Sequence Synthetic peptide
FHKPFKPTHRTL

SEQ ID NO: 68 - protein, 12 aa, Artificial Sequence
  Description of Artificial Sequence Synthetic peptide
QSSCHKHSVRGR

SEQ ID NO: 69 - protein, 12 aa, Artificial Sequence
  Description of Artificial Sequence Synthetic peptide
QSSFSNHSVRRR

SEQ ID NO: 70 - protein, 12 aa, Artificial Sequence
  Description of Artificial Sequence Synthetic peptide
DFDVSFLSARMR

SEQ ID NO: 71 - protein, 8 aa, Artificial Sequence
  Description of Artificial Sequence Synthetic peptide
VLLRRIGG

SEQ ID NO: 72 - protein, 8 aa, Artificial Sequence
  Description of Artificial Sequence Synthetic peptide
HLLRLSEI

SEQ ID NO: 73 - protein, 8 aa, Artificial Sequence
  Description of Artificial Sequence Synthetic peptide
SLLTYMKM

SEQ ID NO: 74 - protein, 8 aa, Artificial Sequence
  Description of Artificial Sequence Synthetic peptide
YLLQKLRN

SEQ ID NO: 75 - protein, 47 aa, Artificial Sequence
  Description of Artificial Sequence Synthetic polypeptide
CSGGASLERIARLEEKVKTLKAQNSELASTANMLREQVAQLKQKGAP

SEQ ID NO: 76 - protein, 49 aa, Artificial Sequence
  Description of Artificial Sequence Synthetic polypeptide
SGASLERIARLEEKVKTLKAQNSELASTANMLREQVAQLKQKGAPSGGC

SEQ ID NO: 77 - protein, 47 aa, Artificial Sequence
  Description of Artificial Sequence Synthetic polypeptide
CSGGASLERIARLEEKVKSFKAQNSENASTANMLREQVAQLKQKGAP

SEQ ID NO: 78 - protein, 49 aa, Artificial Sequence
  Description of Artificial Sequence Synthetic polypeptide
SGASLERIARLEEKVKSFKAQNSENASTANMLREQVAQLKQKGAPSGGC

SEQ ID NO: 79 - protein, 46 aa, Artificial Sequence
  Description of Artificial Sequence Synthetic polypeptide
CSGASLERIARLEEKVKSFKAQNSENASTANMLREQVAQLKQKGAP

SEQ ID NO: 80 - protein, 48 aa, Artificial Sequence
  Description of Artificial Sequence Synthetic polypeptide
GASLERIARLEEKVKTLKAQNSELASTANMLREQVAQLKQKGAPSGGC

SEQ ID NO: 81 - protein, 41 aa, Artificial Sequence
  Description of Artificial Sequence Synthetic polypeptide
ASRELTDTLQAETDQLEDEKSALQTEIANLLKEKEKLEGAP

SEQ ID NO: 82 - protein, 40 aa, Artificial Sequence
  Description of Artificial Sequence Synthetic polypeptide
ASRETDTLQAETDQLEDEKSALQTEIANLLKEKEKLEGAP

SEQ ID NO: 83 - protein, 43 aa, Artificial Sequence
  Description of Artificial Sequence Synthetic polypeptide
SGASRELTDTLQAETDQLEDEKSALQTEIANLLKEKEKLEGAP

SEQ ID NO: 84 - protein, 29 aa, Artificial Sequence
  Description of Artificial Sequence Synthetic peptide
ALKKELQANKKELAQLKWELQALKKELAQ

SEQ ID NO: 85 - protein, 42 aa, Artificial Sequence
  Description of Artificial Sequence Synthetic polypeptide
SGGGSGASALKKELQANKKELAQLKWELQALKKELAQGAPGS

SEQ ID NO: 86 - protein, 30 aa, Artificial Sequence
  Description of Artificial Sequence Synthetic polypeptide
EQLEKKLQALEKKLAQLEWKNQALEKKLAQ

SEQ ID NO: 87 - protein, 20 aa, Artificial Sequence
  Description of Artificial Sequence Synthetic peptide
SGGGSGGGQLGPYELWELSH

SEQ ID NO: 88 - protein, 107 aa, Artificial Sequence
  Description of Artificial Sequence Synthetic polypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 89 - protein, 108 aa, Artificial Sequence
  Description of Artificial Sequence Synthetic polypeptide
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 90 - protein, 10 aa, Artificial Sequence
  Description of Artificial Sequence Synthetic peptide
AGSSAAGSGS

SEQ ID NO: 91 - protein, 18 aa, Artificial Sequence
  Description of Artificial Sequence Synthetic peptide
GGSGGGSGGGSGGGSGGG

SEQ ID NO: 92 - protein, 22 aa, Artificial Sequence
  Description of Artificial Sequence Synthetic peptide
GGSGGGSGGGSGGGSGGGSGGG

SEQ ID NO: 93 - protein, 26 aa, Artificial Sequence
  Description of Artificial Sequence Synthetic peptide
GGSGGGSGGGSGGGSGGGSGGGSGGG

SEQ ID NO: 94 - protein, 15 aa, Artificial Sequence
  Description of Artificial Sequence Synthetic peptide
SGGGGSGGGGSGGGG

SEQ ID NO: 95 - protein, 20 aa, Artificial Sequence
  Description of Artificial Sequence Synthetic peptide
SGGGGSGGGGSGGGGSGGGG

SEQ ID NO: 96 - protein, 25 aa, Artificial Sequence
  Description of Artificial Sequence Synthetic peptide
SGGGGSGGGGSGGGGSGGGGSGGGG

SEQ ID NO: 97 - protein, 5 aa, Artificial Sequence
  Description of Artificial Sequence Synthetic peptide
SGGGS

SEQ ID NO: 98 - protein, 4 aa, Artificial Sequence
  Description of Artificial Sequence Synthetic peptide
SGSG

SEQ ID NO: 99 - protein, 6 aa, Artificial Sequence
  Description of Artificial Sequence Synthetic peptide
SGGGGS

SEQ ID NO: 100 - protein, 5 aa, Artificial Sequence
  Description of Artificial Sequence Synthetic peptide
SGSGG